(19) United States
(12) Patent Application Publication
(10) Pub. No.: US 2006/0041957 A1
(43) Pub. Date: Feb. 23, 2006

(54) Recombinant constructs for use in reducing gene expression

(76) Inventors: Brian McGonigle, Wilmington, DE (US); Johan M. Stoop, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/149,420

(22) Filed: Jun. 9, 2005

Related U.S. Application Data
(60) Provisional application No. 60/578,404, filed on Jun. 9, 2004.

(51) Int. Cl. A01H 1/00; C12N 15/82

(52) U.S. Cl. 800/287; 435/468

(57) ABSTRACT

Recombinant constructs useful for reducing the expression of at least one target nucleic acid fragment of interest and any substantially similar endogenous nucleic acid fragment of interest are disclosed. In particular, a recombinant construct comprising at least one nucleic acid of interest is inserted between two convergent promoters. A plant or plant organ stably transformed with this construct will have a reduction in expression of the target nucleic acid fragments of interest.

This application claims the benefit of U.S. Provisional Application No. 60/578,404, filed Jun. 9, 2004, the entire content of which is herein incorporated by reference.

### FIELD OF THE INVENTION

The field of invention relates to a method for reducing gene expression and, in particular, to recombinant constructs useful for reducing the expression of one or more target endogenous nucleic acid fragments and any substantially similar endogenous nucleic acid fragments.

### BACKGROUND OF THE INVENTION

Plant development is a complex physiological and biochemical process requiring the coordinated expression of many genes. The production of new plant varieties with improved traits can be achieved by modifying this coordinated pattern of gene expression. Recombinant DNA techniques have made it feasible to alter the expression patterns of specific plant genes. In this way, the expression pattern of an individual gene can be either enhanced or diminished in the whole plant, in specific tissues, or in developmental stages.

It is now possible to construct transgenes with defined promoters and terminators and express them in a variety of organisms. There are reports in the literature that some introduced transgenes do not have the expected expression patterns. These unexpected expression patterns are seen in organisms as diverse as nematodes and plants. For example, some plants receiving transgenic copies of an endogenous gene under the control of a strong promoter, sometimes fail to accumulate mRNA for that gene. Furthermore, in some cases all mRNA from endogenous genes having sequence homology to the transgene also fail to accumulate mRNA, effectively eliminating the expression of the endogenous gene product. This was discovered originally when chalcone synthase transgenes in petunia caused suppression of the endogenous chalcone synthase genes (Napoli et al., Plant Cell 2:279-289 (1990)).

The phenomenon observed by Napoli et al. in petunia was referred to as “cosuppression” since expression of both the endogenous gene and the introduced transgene were suppressed (for reviews see Vaucheret et al., Plant J. 16:651-659 (1998); and Gura, Nature 404:804-808 (2000)). Cosuppression technology constitutes the subject matter of U.S. Pat. No. 5,231,020, which issued to Jorgensen et al. on Jul. 27, 1999. In addition to cosuppression, antisense technology has also been used to block the function of specific genes in cells. Antisense RNA is complementary to the normally expressed RNA, and presumably inhibits gene expression by interacting with the normal RNA strand. The mechanisms by which the expression of a specific gene are inhibited by either antisense or sense RNA are on their way to being understood. However, the frequencies of obtaining the desired phenotype in a transgenic plant may vary with the design of the construct, the gene, the strength and specificity of its promoter, the method of transformation and the complexity of transgene insertion events (Baulcombe, Curr. Biol. 12(3):R82-84 (2002); Tang et al., Genes Dev. 17(1):49-63 (2003); Yu et al., Plant Cell. Rep. 22(3):167-174 (2003)). Cosuppression and antisense inhibition are also referred to as “gene silencing”, “post-transcriptional gene silencing” (PTGS), RNA interference or RNAi.

U.S. Patent Publication 2002/0182223 A1, which published Dec. 5, 2002, describes eukaryotic double-stranded RNA (dsRNA) expression vectors containing two promoters directed head-to-head with a designated intervening sequence of interest that appears to be effective in eukaryotic cells, such as a protist cell, containing the dsRNA expression vector, and a vaccine using an attenuated eukaryotic pathogenic cell. Plants and plant organs do not appear to be mentioned. The eukaryotic cells of interest appear to be protozoan parasites that cause diseases, such as, African sleeping sickness, Chagas disease, leishmaniases, toxoplasmosis and malaria.

Bierei et al., (Molecular Breeding 10:107-117 (2002)) tested the effects of convergent transcription on expression levels and analyzed the potential of geminivirus derived DNA sequences to act as bidirectional transcription termination/polyadenylation signals in transgenes to counteract such negative effects. The results appeared to suggest that flanking of a given sequence by two convergent promoters would not be an efficient way to generate double-stranded RNA and induce gene silencing by RNAi.

PCT Publication No. WO 99/53050, which published on Oct. 21, 1999, describes chimeric constructs encoding RNA molecules directed towards a target nucleic acid which are comprised of sense and antisense sequences, such that the expressed RNA is capable of forming an intramolecular double-stranded RNA structure. The expression of these RNA in transgenic organisms results in gene silencing of the homologous target nucleic acid sequences within the cell.

U.S. Pat. No. 5,942,657, issued to Bird et al. on Aug. 25, 1999, and PCT Publication No. WO 93/23551, which published on Nov. 25, 1993, describe coordinated inhibition of plant gene expression in which two or more genes are inhibited by introducing a single control gene having distinct DNA regions homologous to each of the target genes and a promoter operable in plants adapted to transcribe from such distinct regions RNA that inhibits expression of each of the target genes.

The present invention describes the use of recombinant constructs that produce double-stranded RNA, as is discussed below, in ways which heretofore have not been previously described in plants. The double-stranded RNA can be used to efficiently suppress gene expression in plants. The details of this invention are described herein.

### SUMMARY OF THE INVENTION

The present invention concerns a method for reducing expression of at least one target nucleic acid fragment in a plant or plant organ, the method comprising:
(a) stably transforming a plant cell with at least one recombinant construct comprising an isolated nucleic acid fragment of interest situated between a first and second promoter wherein
   (i) the first and second promoters may be the same or different;
   (ii) the first and second promoters have similar spatial and temporal activity; and
   (iii) the first and second promoters are convergent;
   further wherein the recombinant construct is stably integrated into the genome of the plant cell;
(b) regenerating a transformed plant or plant organ from the plant cell of (a); and
(c) evaluating the transformed plant or plant organ for reduced expression of the target nucleic acid fragment when compared to a nontransformed plant or plant organ.

In a first embodiment, this invention concerns A method for reducing expression of at least one target nucleic acid fragment in a plant or plant organ, the method comprising:
(a) stably transforming a plant cell with a recombinant construct comprising a sequence selected from the group consisting of: SEQ ID NO:10, SEQ ID NO:51, SEQ ID NO:52, SEQ ID NO:63, SEQ ID NO:68 and SEQ ID NO:70,
wherein the recombinant construct is stably integrated into the genome of the plant cell;
(b) regenerating a transformed plant or plant organ from the plant cell of (a); and
(c) evaluating the transformed plant or plant organ for reduced expression of the target nucleic acid fragment when compared to a nontransformed plant or plant organ.

In a second embodiment, this invention concerns a recombinant construct for reducing expression of at least one target nucleic acid fragment in a plant cell or plant organ, said construct comprising at lesat one isolated nucleic acid fragment of interest situated between a first and second promoter wherein
(i) the first and second promoters may be the same or different;
(ii) the first and second promoters have similar spatial and temporal activity; and
(iii) the first and second promoters are convergent;
further wherein the recombinant construct is stably integrated into the genome of the plant cell.

In a third embodiment, this invention concerns a transgenic plant or plant organ stably transformed with the recombinant construct of this invention.

In a fourth embodiment, this invention concerns a recombinant construct comprising the sequence set forth in SEQ ID NO:10.

### BIOLOGICAL DEPOSITS

The following plasmids have been deposited with the American Type Culture Collection (ATCC), 10801 University Boulevard, Manassas, Va. 20110-2209, and bears the following designation, Accession Number and date of deposit.

| Plasmid | Accession Number | Date of Deposit |
|---|---|---|
| pKR57 (see FIG. 1) | PTA-6017 | May 28, 2004 |
| pKR63 (see FIG. 2) | PTA-6018 | May 28, 2004 |
| pKR72 (see FIG. 4) | PTA-6019 | May 28, 2004 |
| pKS231 | PTA-6148 | Aug. 4, 2004 |
| pXF1 | 68874 | Dec. 3, 1991 |

### BRIEF DESCRIPTION OF THE DRAWINGS AND SEQUENCE LISTING

The invention can be more fully understood from the following detailed description and the accompanying drawings and Sequence Listing which form a part of this application.

FIG. 1 is a schematic depiction of plasmid pKR57.

FIG. 2 is a schematic depiction of plasmid pKR63.

FIG. 3 is a schematic depiction of plasmid pDS1.

FIG. 4 is a schematic depiction of plasmid pKR72.

FIG. 5 is a schematic depiction of plasmid pDS2.

FIG. 6 is a schematic depiction of plasmid pDS3 (orientation 1).

FIG. 7 is a schematic depiction of plasmid pDS3 (orientation 2).

FIG. 8 is a schematic depiction of plasmid pDS5.

FIG. 9 is a schematic depiction of plasmid pJMS10.

FIG. 10 is a schematic depiction of plasmid SH60.

FIG. 11 is a thin layer chromatography (TLC) analysis of individual somatic embryos transformed with a construct targeted for silencing of multiple galactinol synthase genes. As shown in FIG. 11, thirteen out of fifteen embryos show reduced levels of raffinose sugars (raffinose and stachyose) when compared to a to wild-type soybean (Jack).

FIG. 12 is a schematic depiction of plasmid PHP22905.

FIG. 13 is a schematic depiction of plasmid PHP22972.

SEQ ID NO:1 is the 4479 bp sequence of pKR57.

SEQ ID NO:2 is the 5010 bp sequence of pKR63.

SEQ ID NO:3 is the 5414 bp sequence of pDS1.

SEQ ID NO:4 is the 7085 bp sequence of pKR72.

SEQ ID NO:5 is the 5303 bp sequence of pDS2.

SEQ ID NO:6 is the 8031 bp sequence of pDS3 (orientation 1).

SEQ ID NO:7 is the 8031 bp sequence of pDS3 (orientation 2).

SEQ ID NO:8 is the sequence of an oligonucleotide primer used in a PCR amplification of the soybean Fad2-1 gene for insertion into plasmid pDS3 to produce plasmid pDS5.

SEQ ID NO:9 is the sequence of an oligonucleotide primer used in a PCR amplification of the soybean Fad2-1 gene for insertion into plasmid pDS3 to produce plasmid pDS5.

SEQ ID NO:10 is the 8642 bp sequence of pDS5.

SEQ ID NO:11 is the sequence of soybean seed galactinol synthase cDNA (GAS3).

SEQ ID NO:12 is the sequence of soybean seed galactinol synthase cDNA (GAS1). Nucleotide 1 is the first nucleotide following the Pst I restriction site, reading from 5′ to 3′ on the cDNA insert, nucleotide 1406 is the last nucleotide of the cDNA insert, immediately before the first nucleotide of the Kpn I restriction site of plasmid pS21. Nucleotides 1 to 138 are the 5′ untranslated sequence, nucleotides 139 to 141 are the translation initiation codon, nucleotides 1123 to 1125 are the termination codon, and nucleotides 1126 to 1406 are the 3′ untranslated sequence.

SEQ ID NO:13 is the sequence of soybean seed galactinol synthase cDNA (GAS2) (found in clone ses4d.pk0017.b8).

SEQ ID NO:14 is the sequence of the GAS1 oligonucleotide primer designed to add a Not I restriction endonuclease site at the 5′ end.

SEQ ID NO:15 is the sequence of the GAS1 oligonucleotide primer designed to add a stop codon (TGA) and an Xho I restriction endonuclease site at the 3′ end.

SEQ ID NO:16 is the DNA sequence comprising the 519 bp sequence from soybean GAS1 resulting from the GAS1 oligonucleotides primers of SEQ ID NO:14 and SEQ ID NO:15.

SEQ ID NO:17 is the sequence of the GAS2 oligonucleotide primer designed to add a Xho I restriction endonuclease site at the 5′ end.

SEQ ID NO:18 is the sequence of the GAS2 oligonucleotide primer designed to add a stop codon (TAA) and a Pst I restriction endonuclease site at the 3′ end.

SEQ ID NO:19 is the DNA sequence comprising the 519 bp sequence from soybean GAS2 resulting from the GAS2 oligonucleotides primers of SEQ ID NO:17 and SEQ ID NO:18.

SEQ ID NO:20 is the sequence of the GAS3 oligonucleotide primer designed to add a Pst I restriction endonuclease site at the 5′ end.

SEQ ID NO:21 is the sequence of the GAS3 oligonucleotide primer designed to add a stop codon (TAG) and a Not I restriction endonuclease site at the 3′ end.

SEQ ID NO:22 is the DNA sequence comprising the 519 bp sequence from soybean GAS3 resulting from the GAS3 oligonucleotides primers of SEQ ID NO:20 and SEQ ID NO:21.

SEQ ID NO:23 is the 6383 bp sequence obtained by Kpn I digestion of pKS231.

SEQ ID NO:24 is the deduced amino acid sequence of the mutant soybean acetolactate synthase (ALS) gene found in Example 6.

SEQ ID NO:25 is the 1585 bp sequence comprising the partial sequences of GAS1 (SEQ ID NO:16), GAS2 (SEQ ID NO:19) and GAS3 (SEQ ID NO:22).

SEQ ID NO:26 is the 8966 bp sequence of pKS210.

SEQ ID NO:27 is the sequence of the oligonucleotide primer BM1 used in a PCR amplification of a fragment of pKS210.

SEQ ID NO:28 is the sequence of the oligonucleotide primer BM2 used in a PCR amplification of a fragment of pKS210.

SEQ ID NO:29 is the 8911 bp sequence of pDN10.

SEQ ID NO:30 is the 890 bp sequence of recombinant DNA fragment KSFAD2-hybrid.

SEQ ID NO:31 is the sequence of the oligonucleotide primer KS1 used in a PCR amplification of a fragment of the FAD2-2 gene.

SEQ ID NO:32 is the sequence of the oligonucleotide primer KS2 used in a PCR amplification of a fragment of the FAD2-2 gene.

SEQ ID NO:33 is the sequence of the oligonucleotide primer KS3 used in a PCR amplification of a fragment of the FAD2-1 gene.

SEQ ID NO:34 is the sequence of the oligonucleotide primer KS4 used in a PCR amplification of a fragment of the FAD2-1 gene.

SEQ ID NO:35 is the 4351 bp sequence of recombinant DNA fragment 1028.

SEQ ID NO:36 is the 50 bp sequence that of the longest stretch of continuous identical nucleotides shared by LOX1 and LOX2.

SEQ ID NO:37 is the 9256 bp sequence of pDS8.

SEQ ID NO:38 is the 12388 bp sequence of plasmid PHP21676.

SEQ ID NO:39 is the 3414 bp sequence constructed by PCR amplification in Example 6E.

SEQ ID NO:40 is the sequence of the oligonucleotide primer BM3 used in a PCR amplification of the approximately 0.9 kb DNA fragment, comprising a portion of the soybean FAD2-2 gene and a portion of the soybean FAD2-1 gene and it was also used in a PCR amplification of a mixture of the approximately 1.5 kb DNA fragment, comprising a portion of the soybean FAD2-2 gene and a portion of the soybean FAD2-1 gene, and the approximately 0.65 kb fragment, comprising a portion of a FAD3 gene.

SEQ ID NO:41 is the sequence of the oligonucleotide primer BM4 used in a PCR amplification of the approximately 0.9 kb DNA fragment, comprising a portion of the soybean FAD2-2 gene and a portion of the soybean FAD2-1 gene.

SEQ ID NO:42 is the sequence of the oligonucleotide primer BM5 used in a PCR amplification of the approximately 0.65 kb DNA fragment, comprising a portion of the soybean FAD3 gene.

SEQ ID NO:43 is the sequence of the oligonucleotide primer BM6 used in a PCR amplification of the approximately 0.65 kb DNA fragment, comprising a portion of the soybean FAD3 gene.

SEQ ID NO:44 is the sequence of the oligonucleotide primer BM7 used in a PCR amplification of a mixture of the approximately 1.5 kb DNA fragment, comprising a portion of the soybean FAD2-2 gene and a portion of the soybean FAD2-1 gene, and the approximately 0.65 kb DNA fragment, comprising a portion of a FAD3 gene.

SEQ ID NO:45 is the sequence of the oligonucleotide primer BM8 used in a PCR amplification of an approximately 1.9 kb DNA fragment, comprising portions of the LOX2 and LOX3 genes.

SEQ ID NO:46 is the sequence of the oligonucleotide primer BM9 used in a PCR amplification of the approximately 1.9 kb DNA fragment, comprising portions of the LOX2 and LOX3 genes.

SEQ ID NO:47 is the 2917 bp sequence of se4.pk0007.e7 which encodes soybean LOX2.

SEQ ID NO:48 is the 2794 bp sequence of sgs1c.pk0002.g4 which encodes soybean LOX3.

SEQ ID NO:49 is the 12678 bp sequence of plasmid PHP22905.

SEQ ID NO:50 is the 12678 bp sequence of plasmid PHP22972.

SEQ ID NO:51 is the 10164 bp sequence of recombinant DNA fragment PHP22905A.

SEQ ID NO:52 is the 10164 bp sequence of recombinant DNA fragment PHP22972A.

SEQ ID NO:53 is the amino acid sequence of Euphorbia lagascae CYP726A1 (NCBI Accession No. AAL62063.1; NCBI General Identifier No. 18157659).

SEQ ID NO:54 is the 1784 bp sequence of the entire cDNA insert in clone sfl1.pk0045.g7.

SEQ ID NO:55 is the deduced amino acid sequence obtained from translating nucleotides 22 through 1548 of SEQ ID NO:54.

SEQ ID NO:56 is the sequence of the oligonucleotide primer BM10 used in a PCR amplification of the approximately 1100 bp fragment, comprising a portion of the P450-EPOX gene.

SEQ ID NO:57 is the sequence of the oligonucleotide primer BM11 used in a PCR amplification of the approximately 1100 bp fragment, comprising a portion of the P450-EPOX gene.

SEQ ID NO:58 is the sequence of the oligonucleotide primer BM12 used in a PCR amplification of the approximately 1880 bp fragment, comprising portions of the LOX2 and LOX3 genes.

SEQ ID NO:59 is the sequence of the oligonucleotide primer BM13 used in a PCR amplification of the approximately 1880 bp fragment, comprising portions of the LOX2 and LOX3 genes, and it was also used in a PCR amplification of the approximately 3420 bp fragment, comprising a portion of the FAD2-2 gene, a portion of the FAD2-1 gene, a portion of the FAD3 gene, and portions of the LOX2 and LOX3 genes.

SEQ ID NO:60 is the 3001 bp sequence comprising a portion of the P450-EPOX gene and portions of the LOX2 and LOX3 genes.

SEQ ID NO:61 is the 6914 bp sequence comprising SEQ ID NO:60 cloned into pPCR2.1.

SEQ ID NO:62 is the 12249 bp sequence of plasmid PHP23466.

SEQ ID NO:63 is the 9735 bp sequence of recombinant DNA fragment PH P23466A.

SEQ ID NO:64 is the 5031 bp sequence comprising a 1100 bp fragment of clone sfl1.pk0045.g7 inserted into plasmid pCR2.1.

SEQ ID NO:65 is the sequence of the oligonucleotide primer BM15 used in a PCR amplification of the approximately 3420 bp fragment, comprising a portion of the FAD2-2 gene, a portion of the FAD2-1 gene, a portion of the FAD3 gene, and portions of the LOX2 and LOX3 genes.

SEQ ID NO:66 is the 7341 bp sequence comprising a portion of the FAD2-2 gene, a portion of the FAD2-1 gene, a portion of the FAD3 gene, and portions of the LOX2 and LOX3 genes inserted into plasmid pCR2.1.

SEQ ID NO:67 is the 13788 bp sequence of plasmid PHP23465.

SEQ ID NO:68 is the 11274 bp sequence of recombinant DNA fragment PHP23465A.

SEQ ID NO:69 is the 8031 bp sequence of resulting from digestion of pDS3 (orientation 2) with Not 1.

SEQ ID NO:70 is the 9616 bp sequence of plasmid SH60.

### DETAILED DESCRIPTION OF THE INVENTION

The disclosure of all patents, patent applications and/or any non-patent references referred to herein are incorporated by reference.

A number of terms shall be utilized in the context of this disclosure.

The term “target nucleic acid fragment” refers to any nucleic acid fragment whose expression in a plant or plant organ is to be reduced. Thus, the “target nucleic acid fragment” is a nucleic acid fragment, preferably an endogenous nucleic acid fragment, whose expression is modulated (reduced or suppressed) by a recombinant construct of the invention that is stably integrated into the genome of the plant or plant organ as discussed herein.

The term “isolated nucleic acid fragment of interest” refers to the nucleic acid fragment in the recombinant construct situated between the first and second promoters. The isolated nucleic acid fragment of interest is chosen or designed based on the target nucleic acid fragment or fragments whose expression is to be reduced. The isolated nucleic acid fragment of interest can be a single sequence or can comprise multiple sequences and is more fully discussed below.

The term “plant organ” refers to plant tissue or group of tissues that constitute a morphologically and functionally distinct part of a plant. The term “genome” refers to the following: 1. The entire complement of genetic material (genes and non-coding sequences) is present in each cell of an organism, or virus or organelle. 2. A complete set of chromosomes inherited as a (haploid) unit from one parent. The term “stably integrated” refers to the transfer of a nucleic acid fragment into the genome of a host organism or cell resulting in genetically stable inheritance. The term “chimera” refers to an organism such as a plant that is composed of tissue of more than one genotype. For example, a plant is said to be a chimera when cells of more than one genotype are present in the plant, e.g., some chimeras cause a visual variegation.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of chimeric genes to produce the desired phenotype in a transformed plant. Chimeric genes can be designed for use in co-suppression or antisense by linking a nucleic acid fragment or subfragment thereof, whether or not it encodes an active enzyme, in the appropriate orientation relative to a plant promoter sequence.

The terms “homology”, “homologous”, “substantially similar” and “corresponding substantially” are used interchangeably herein. They refer to nucleic acid fragments wherein changes in one or more nucleotide bases does not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

Moreover, the skilled artisan recognizes that substantially similar nucleic acid sequences encompassed by this invention are also defined by their ability to hybridize, under moderately stringent conditions (for example, 0.5×SSC, 0.1% SDS, 60° C.) with the sequences exemplified herein, or to any portion of the nucleotide sequences reported herein and functional equivalents thereof. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions involves a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions involves the use of higher temperatures in which the washes are identical to those above except for the temperature of the final two 30 min washes in 0.2×SSC, 0.5% SDS was increased to 60° C. Another preferred set of highly stringent conditions involves the use of two final washes in 0.1×SSC, 0.1% SDS at 65° C.

With respect to the degree of substantial similarity between the target nucleic acid fragment (endogenous nucleic acid fragment) and the region in the recombinant construct having homology to the target mRNA, such sequences should be at least 25 nucleotides in length, preferably at least 50 nucleotides in length, more preferably at least 100 nucleotides in length, again more preferably at least 200 nucleotides in length, and most preferably at least 300 nucleotides in length; and should be at least 80% identical, preferably at least 85% identical, more preferably at least 90% identical, and most preferably at least 95% identical, or any integer percentage from 80% to 100%.

It is well understood by one skilled in the art that many levels of sequence identity are useful in identifying related polypeptide sequences. Useful examples of percent identities are 50%, 55%, 60%, 65%, 70%, 75%, 80%, 85%, 90%, or 95%, or any integer percentage from 55% to 100%. Sequence alignments and percent similarity calculations may be determined using a variety of comparison methods designed to detect homologous sequences including, but not limited to, the Megalign program of the LASARGENE bioinformatics computing suite (DNASTAR Inc., Madison, Wis.). Multiple alignment of the sequences are performed using the Clustal method of alignment (Higgins and Sharp, CABIOS. 5:151-153 (1989)) with the default parameters (GAP PENALTY=10, GAP LENGTH PENALTY=10). Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4.“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Thus, promoters can have activity with similar spatial and temporal patterns of expression or different spatial and temporal patterns of expression. The terms “spatial and temporal” and “spatiotemporal” are used interchangeably and relate to space and time. Promoters which cause a gene to be expressed in most cell types at most times are commonly referred to as “constitutive promoters”. New promoters of various types useful in plant cells are constantly being discovered; numerous examples may be found in the compilation by Okamuro and Goldberg, Biochemistry of Plants 15:1-82 (1989). It is further recognized that since in most cases the exact boundaries of regulatory sequences have not been completely defined, DNA fragments of some variation may have identical promoter activity.

“Convergent promoters” refers to promoters that are situated on either side of the isolated nucleic acid fragment of interest such that the direction of transcription from each promoter is opposing each other.

An “intron” is an intervening sequence in a gene that does not encode a portion of the protein sequence. Thus, such sequences are transcribed into RNA but are then excised and are not translated. The term is also used for the excised RNA sequences. An “exon” is a portion of the sequence of a gene that is transcribed and is found in the mature messenger RNA derived from the gene, but is not necessarily a part of the sequence that encodes the final gene product.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D., Molecular Biotechnology 3:225 (1995)).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., Plant Cell 1:671-680 (1989).

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a DNA that is complementary to and synthesized from a mRNA template using the enzyme reverse transcriptase. The cDNA can be single-stranded or converted into the double-stranded form using the Klenow fragment of DNA polymerase 1. “Sense” RNA refers to RNA transcript that includes the mRNA and can be translated into protein within a cell or in vitro. “Antisense RNA” refers to an RNA transcript that is complementary to all or part of a target primary transcript or mRNA and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “non-naturally occurring” means artificial, not consistent with what is normally found in nature.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

The term “expression”, as used herein, refers to the production of a functional end-product. “Antisense inhibition” refers to the production of antisense RNA transcripts capable of suppressing the expression of the target protein. “Co-suppression” refers to the production of sense RNA transcripts capable of suppressing the expression of identical or substantially similar foreign or endogenous genes (U.S. Pat. No. 5,231,020).

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

“Stable transformation” refers to the transfer of a nucleic acid fragment into a genome of a host organism, including either nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms. The preferred method of cell transformation of rice, corn and other monocots is the use of particle-accelerated or “gene gun” transformation technology (Klein et al., Nature (London) 327:70-73 (1987); U.S. Pat. No. 4,945,050), or an Agrobacterium-mediated method using an appropriate Ti plasmid containing the transgene (Ishida Y. et al., Nature Biotech. 14:745-750 (1996)).

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

The term “recombinant” refers to an artificial combination of two otherwise separated segments of sequence, e.g., by chemical synthesis or by the manipulation of isolated segments of nucleic acids by genetic engineering techniques.

The term “host” refers to any organism, or cell thereof, whether human or non-human into which a recombinant construct can be stably or transiently introduced in order to reduce gene expression.

“PCR” or “Polymerase Chain Reaction” is a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

The terms “recombinant construct”, “expression construct” and “recombinant expression construct” are used interchangeably herein. Such construct may be itself or may be used in conjunction with a vector. A “vector” is a DNA molecule that can be replicated in a cell and that can serve as the vehicle for transfer to such a cell of DNA that has been inserted into it by recombinant techniques. If a vector is used then the choice of vector is dependent upon the method that will be used to transform host plants as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by Northern analysis of mRNA expression, Western analysis of protein expression, or phenotypic analysis.

Co-suppression constructs in plants previously have been designed by focusing on overexpression of a nucleic acid sequence having homology to an endogenous mRNA, in the sense orientation, which results in the reduction of all RNA having homology to the overexpressed sequence (see Vaucheret et al., Plant J. 16:651-659 (1998); and Gura, Nature 404:804-808 (2000)). Recent work has described the use of “hairpin” structures that incorporate all, or part, of an mRNA encoding sequence in a complementary orientation that results in a potential “stem-loop” structure for the expressed RNA (PCT Publication No. WO 99/53050, which published on Oct. 21, 1999). This increases the frequency of co-suppression in the recovered transgenic plants. Another variation describes the use of plant viral sequences to direct the suppression, or “silencing”, of proximal mRNA encoding sequences (PCT Publication No. WO 98/36083, which published on Aug. 20, 1998; U.S. Pat. No. 6,635,805, issued to Angell et al. on Oct. 21, 2003). PCT Publication No. WO 02/00904, which published on Jan. 3, 2002, the disclosure of which is hereby incorporated by reference in its entirety, describes single, or multiple, gene co-suppression in an invertebrate host.

The present invention is concerned with the ability to efficiently reduce or suppress the expression of at least one target nucleic acid fragment in a plant or plant organ. The target nucleic acid can be any coding or non-coding region in the plant genome. For this purpose, a recombinant construct could be prepared that would be capable of reducing or suppressing expression of a gene in a plant such as soybean. The present invention concerns a method for reducing expression of at least one target mRNA in a plant or plant organ, the method comprising:
(a) stably transforming a plant cell with a recombinant construct comprising at least one isolated nucleic acid fragment of interest situated between a first and second promoter wherein
   (i) the first and second promoters may be the same or different;
   (ii) the first and second promoters have similar spatial and temporal activity; and
   (iii) the first and second promoters are convergent;
   further wherein the recombinant construct is stably integrated into the genome of the plant cell;
(b) regenerating a transformed plant or plant organ from the plant cell of (a); and
(c) evaluating the transformed plant or plant organ for reduced expression of the target nucleic acid fragment when compared to a nontransformed plant or plant organ.

In another aspect this invention concerns A method for reducing expression of at least one target nucleic acid fragment in a plant or plant organ, the method comprising:
(a) stably transforming a plant cell with a recombinant construct comprising a sequence selected from the group consisting of: SEQ ID NO:10, SEQ ID NO:51, SEQ ID NO:52, SEQ ID NO:63, SEQ ID NO:68 and SEQ ID NO:70,
wherein the recombinant construct is stably integrated into the genome of the plant cell;
(b) regenerating a transformed plant or plant organ from the plant cell of (a); and
(c) evaluating the transformed plant or plant organ for reduced expression of the target nucleic acid fragment when compared to a nontransformed plant or plant organ.

Examples of a target nucleic acid fragment whose expression can be reduced or suppressed include, but are not limited to, one or more nucleic acid fragments involved in primary metabolism, more specifically those involved in cell wall biosynthesis, membrane biosynthesis, amino acid and protein biosynthesis, nucleic acid biosynthesis, carbohydrate biosynthesis, cytoskeleton biosynthesis or photosynthesis, as well as nucleic acid fragments involved with encoding transcription factors, those involved in abiotic stress response, those involved in biotic stress response, those involved in senescence and programmed death, those involved in the molecular physiology of mineral nutrient acquisition, transport and utilization, those involved in signal perception and development, those involved in nitrogen or sulfur metabolism, those involved in reproductive development, those involved in the basic development or elaboration of the plant form or those involved in the biosynthesis of hormones and elicitor molecules. Suitable target nucleic acid fragments can also be involved with secondary metabolism more specifically those involved in the biosynthesis of terpenoids, alkaloids, or phenylpropanoids. There also can be mentioned as the target nucleic acid fragment those nucleic acid fragments encoding lipoxygenases, fatty acid biosynthesis enzymes, carotenoid biosynthetic enzymes, related-to carotenoid dioxygenase enzymes, beta-amyrin synthase, oxidosqualene cyclases, hydroperoxide lyases, lipid oxidation enzymes, aureusidin synthase, polyphenol oxidases, isoflavone synthase, dihydroflavonol reductase, flavonol synthase, chalcone reductase, or chalcone isomerase.

The recombinant construct of this invention for reducing expression of at least one target nucleic acid fragment in a plant cell or plant organ, said construct comprising at least one isolated nucleic acid fragment of interest situated between a first and second promoter wherein
(i) the first and second promoters may be the same or different;
(ii) the first and second promoters have similar spatial and temporal activity; and
(iii) the first and second promoters are convergent;
further wherein the recombinant construct is stably integrated into the genome of the plant cell.

The isolated nucleic acid of interest, that is situated between the first and second promoters, can be any portion of the gene, such as a coding or non-coding region, wherein the entire gene specifies both regulatory and sequence information for the target sequence of interest, so long as the isolated nucleic acid of interest is related by sequence homology to the target nucleic acid fragment of interest whose expression is targeted for being reduced. Thus, the isolated nucleic acid of interest used in making the recombinant construct of the invention need not be identical to the target nucleic acid fragment. The isolated nucleic acid of interest just needs to share enough homology to be useful in reducing expression of the target nucleic acid fragment.

With respect to the degree of substantial similarity between the target nucleic acid fragment (endogenous nucleic acid fragment) and the region in the recombinant construct having homology or sequence identity to the target mRNA, such sequences should be at least 25 nucleotides in length, preferably at least 50 nucleotides in length, more preferably at least 100 nucleotides in length, again more preferably at least 200 nucleotides in length, and most preferably at least 300 nucleotides in length; and should be at least 80% identical, preferably at least 85% identical, more preferably at least 90% identical, and most preferably at least 95% identical, or any integer percentage from 80% to 100%.

Furthermore, it is believed that the isolated nucleic acid of interest can be used to reduce or suppress expression of more than one target nucleic acid fragment. It has been suggested that double stranded RNA formed in cis that is homologous to multiple genes is effective in suppressing those multiple genes. (Custom Knock-Outs with Hairpin RNA-Mediated Gene Silencing. Wesley, Susan Varsha; Liu, Qing; Wielopolska, Anna; Ellacott, Geoff; Smith, Neil; Singh, Surinder; Helliwell, Chris. Plant Functional Genomics: Methods and Protocols. August 2003 pps. 273-286). As such, it is believed that multiple isolated nucleic acids of interest operably linked between one set of convergent promoters should be able to reduce expression of multiple target nucleic acid fragments that are not related by homology or share low sequence identity.

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In: Methods for Plant Molecular Biology, (Eds.), Academic Press, Inc., San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention comprising the desired “recombinant construct” is cultivated using methods well known to one skilled in the art.

In addition to the above discussed procedures, those skilled in the art are familiar with the standard resource materials which describe specific conditions and procedures for the construction, manipulation and isolation of macromolecules (e.g., DNA molecules, plasmids, etc.), generation of recombinant DNA fragments and recombinant expression constructs and the screening and isolating of clones, (see for example, Sambrook et al. (1989) Molecular Cloning: A Laboratory Manual, Cold Spring Harbor Press; Maliga et al. (1995) Methods in Plant Molecular Biology, Cold Spring Harbor Press; Birren et al. (1998) Genome Analysis: Detecting Genes, 1, Cold Spring Harbor, N.Y.; Birren et al. (1998) Genome Analysis: Analyzing DNA, 2, Cold Spring Harbor, N.Y.; Plant Molecular Biology: A Laboratory Manual, eds. Clark, Springer, New York (1997)).

Any promoter useful in plant transgene expression can be used to practice the invention. The promoters can be the same or different. The promoters are convergent with the isolated nucleic acid fragment being situated between the convergent promoters. It is important that the promoters have similar spatial and temporal activity, i.e., similar spatial and temporal patterns of expression, so that double-stranded RNA is produced in plants or plant organs by the recombinant construct that is stably integrated into the genome of the plant or plant organ.

There can be mentioned a β-conglycinin promoter, a Kunitz soybean trypsin inhibitor (abbreviated as KSTI, Kti or KTi3) promoter, a napin promoter, beta-phaseolin promoter, oleosin promoter, albumin promoter, a zein promoter, a Bce4 promoter, a legumin B4 promoter, a T7 promoter and a ³⁵S promoter. The preferred promoters are that of the α′-subunit of β-conglycinin (referred to herein as the β-conglycinin promoter) and a Kunitz soybean trypsin inhibitor (abbreviated as KSTI, Kti or KTi3) promoter. Particularly preferred promoters are those that allow seed-specific expression. This may be especially useful since seeds are the primary source of consumable protein and oil, and also since seed-specific expression will avoid any potential deleterious effect in non-seed tissues.

Co-suppressed plants that comprise recombinant expression constructs with the promoter of the α′-subunit of β-conglycinin will often exhibit suppression of both the α and α′ subunits of beta-congylcinin (as described in PCT Publication No. WO 97/47731, which published on Dec. 18, 1997, the disclosure of which is hereby incorporated by reference).

Examples of seed-specific promoters include, but are not limited to, the promoters of seed storage proteins, which can represent up to 90% of total seed protein in many plants. The seed storage proteins are strictly regulated, being expressed almost exclusively in seeds in a highly tissue-specific and stage-specific manner (Higgins et al., Ann. Rev. Plant Physiol. 35:191-221 (1984); Goldberg et al., Cell 56:149-160 (1989)). Moreover, different seed storage proteins may be expressed at different stages of seed development.

Expression of seed-specific genes has been studied in great detail (See reviews by Goldberg et al., Cell 56:149-160 (1989) and Higgins et al., Ann. Rev. Plant Physiol. 35:191-221 (1984)). There are currently numerous examples of seed-specific expression of seed storage protein genes in transgenic dicotyledonous plants. These include genes from dicotyledonous plants for bean β-phaseolin (Sengupta-Gopalan et al., Proc. Natl. Acad. Sci. USA 82:3320-3324 (1985); Hoffman et al., Plant Mol. Biol. 11:717-729 (1988)), bean lectin (Voelker et al., EMBO J. 6:3571-3577 (1987)), soybean lectin (Okamuro et al., Proc. Natl. Acad. Sci. USA 83:8240-8244 (1986)), soybean Kunitz trypsin inhibitor (Perez-Grau et al., Plant Cell 1:1095-1109 (1989)), soybean β-conglycinin (Beachy et al., EMBO J. 4:3047-3053 (1985); pea vicilin (Higgins et al., Plant Mol. Biol. 11:683-695 (1988)), pea convicilin (Newbigin et al., Planta 180:461470 (1990)), pea legumin (Shirsat et al., Mol. Gen. Genetics 215:326-331 (1989)); rapeseed napin (Radke et al., Theor. Appl. Genet. 75:685-694 (1988)) as well as genes from monocotyledonous plants such as for maize 15 kD zein (Hoffman et al., EMBO J. 6:3213-3221 (1987)), maize 18 kD oleosin (Lee at al., Proc. Natl. Acad. Sci. USA 88:6181-6185 (1991)), barley β-hordein (Marris et al., Plant Mol. Biol. 10:359-366 (1988)) and wheat glutenin (Colot et al., EMBO J. 6:3559-3564 (1987)). Moreover, promoters of seed-specific genes operably linked to heterologous coding sequences in chimeric gene constructs also maintain their temporal and spatial expression pattern in transgenic plants. Such examples include use of Arabidopsis thaliana 2S seed storage protein gene promoter to express enkephalin peptides in Arabidopsis and Brassica napus seeds (Vandekerckhove et al., Bio/Technology 7:929-932 (1989)), bean lectin and bean β-phaseolin promoters to express luciferase (Riggs et al., Plant Sci. 63:47-57 (1989)), and wheat glutenin promoters to express chloramphenicol acetyl transferase (Colot et al., EMBO J. 6:3559-3564 (1987)).

As was noted above, any type of promoter such as constitutive, tissue-preferred, inducible promoters can be used to practice the invention. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the GRP1-8 promoter and other transcription initiation regions from various plant genes known to those of skill.

Examples of inducible promoters are the Adh1 promoter which is inducible by hypoxia or cold stress, the Hsp70 promoter which is inducible by heat stress, and the PPDK promoter which is inducible by light. Also useful are chemical-inducible promoters whose transcriptional activity is regulated by the presence or absence of alcohol, tetracycline, steroids (i.e., ecdysone; U.S. Pat. No. 6,379,945), metals and other compounds.

Examples of promoters under developmental control include promoters that initiate transcription preferentially in certain tissues, such as leaves, roots, fruit, seeds, or flowers. One such example is the RuBis Co promoter. Another exemplary promoter is the anther specific promoter 5126 (U.S. Pat. Nos. 5,689,049 and 5,689,051). In addition to those mentioned above, other examples of seed-specific promoters include, but are not limited to, 27 kD gamma zein promoter and waxy promoter (Boronat, A., Martinez, M. C., Reina, M., Puigdomenech, P. and Palau, J.; Isolation and sequencing of a 28 kD glutelin-2 gene from maize: Common elements in the 5′ flanking regions among zein and glutelin genes; Plant Sci. 47:95-102 (1986) and Reina, M., Ponte, I., Guillen, P., Boronat, A. and Palau, J., Sequence analysis of a genomic clone encoding a Zc2 protein from Zea mays W64 A, Nucleic Acids Res. 18(21):6426 (1990)). See the following reference relating to the waxy promoter: Kloesgen, R. B., Gierl, A., Schwarz-Sommer, Z. S. and Saedler, H., Molecular analysis of the waxy locus of Zea mays, Mol. Gen. Genet. 203:237-244 (1986). Promoters that express in the embryo, pericarp, and endosperm are disclosed in PCT Publication No. WO 00/11177, which published Mar. 2, 2000, and PCT Publication No. WO 00/12733, which published Mar. 9, 2000. The disclosures of each of these are incorporated herein by reference in their entirety.

Either heterologous or non-heterologous (i.e., endogenous) promoters can be used to practice the invention.

The promoter is then operably linked using conventional means well known to those skilled in the art to a DNA sequence which, when expressed by a host produces an RNA meeting certain criteria.

Any plant or plant organ, into which the recombinant construct of this invention can be stably integrated in order to alter gene expression may be used. The plant may be a monocot, dicot or gymnosperm.

Examples of suitable plants which can be used to practice the invention include, but are not limited to, soybean, corn, alfalfa, canola, sorghum, sunflower, wheat, rice, oat, cotton, rye, sorghum, sugarcane, tomato, tobacco, millet, flax, potato, barley, Arabidopsis, bean, pea, rape, safflower, asparagus, beet, broccoli, cabbage, carrot, cauliflower, celery, cucumber, eggplant, lettuce, onion, pepper, potato, pumpkin, radish, spinach, squash, taro, tomato, zucchini, almond, apple, apricot, banana, blackberry, blueberry, cacao, cherry, coconut, cranberry, date, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, watermelon, etc.

Evaluation of reduced expression of a target nucleic acid fragment in a plant or plant organ, may be accomplished by a variety of means such as Northern analysis of mRNA expression, Western analysis of protein expression, or phenotypic analysis. Seed size, leaf color, saponin levels, isoflavone levels and carotenoid levels are examples of phenotypic traits found in plants. Expression products of a target nucleic acid fragment can be detected in any of a variety of ways, depending upon the nature of the product (e.g., Western blot and enzyme assay). Once transgenic plants have been obtained, they may be grown to produce plant tissues or parts having the desired phenotype. The plant tissue or plant parts may be harvested, and/or the seed collected. The seed may serve as a source for growing additional plants with tissues or parts having the desired characteristics.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Those skilled in the art will appreciate that plasmids are circular molecules and position 1 of its sequence is artificially set. The disclosures contained within the references used herein are hereby incorporated by reference.

### Example 1

### Preparation of Recombinant Constructs

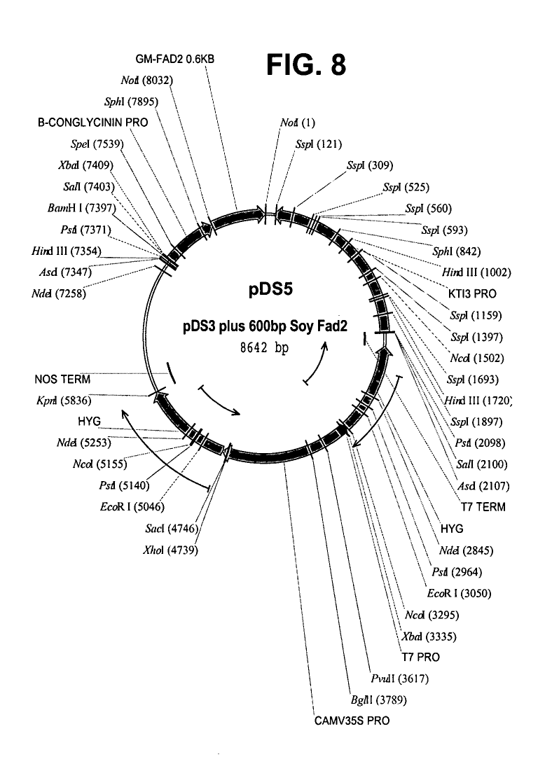

The following example describes the preparation of a recombinant construct (vector pDS5; FIG. 8) that would be capable of suppressing expression of a gene in soybeans, Glycine max.

Fad2-1 was selected as the nucleic acid fragment of interest. Fad2-1 is described in PCT Publication No. WO 94/11516, which published on May 26, 1994. Fad2-1 is a gene locus encoding a A¹² desaturase from soybean that introduces a double bond into the oleic acid chain to form a polyunsaturated fatty acid. A delta-12 desaturase refers to a fatty acid desaturase that catalyzes the formation of a double bond between carbon positions 6 and 7 (numbered from the methyl end), (i.e., those that correspond to carbon positions 12 and 13 (numbered from the carbonyl carbon) of an 18 carbon-long fatty acyl chain).

Reduction in the expression of Fad2-1 results in the accumulation of oleic acid (18:1, or an 18 carbon fatty acid tail with a single double bond) and a corresponding decrease in polyunsaturated fatty acid content. The methods used to make pDS5 are described below.

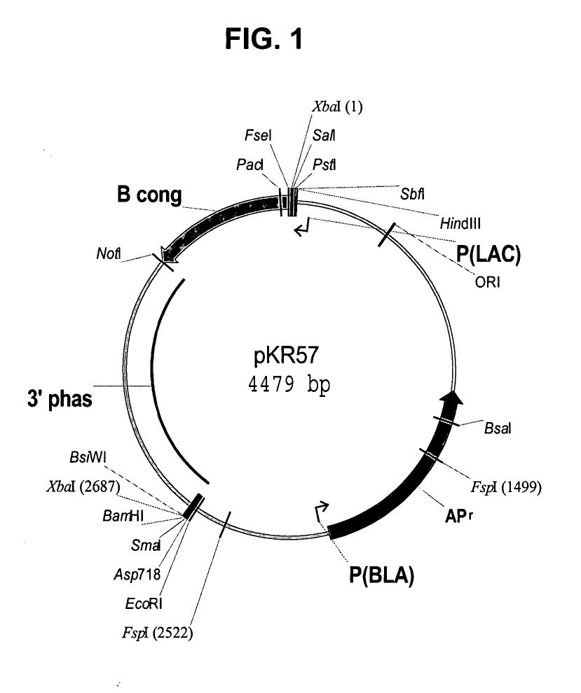

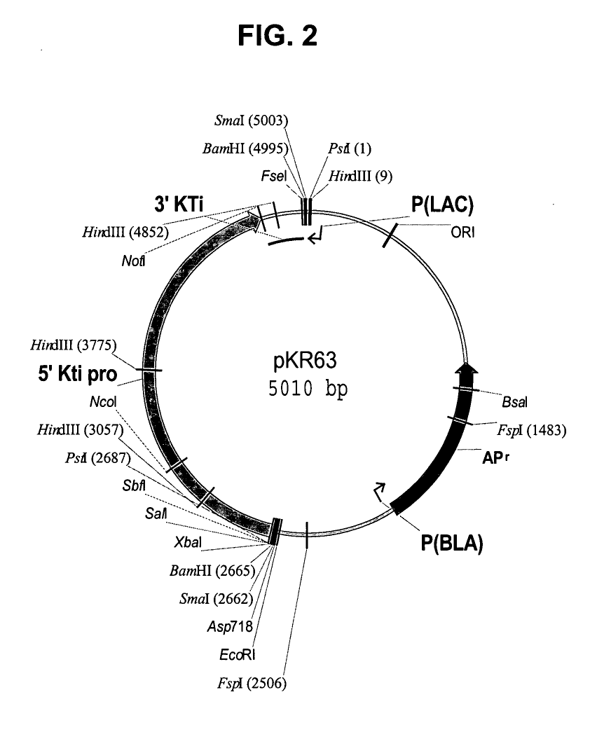

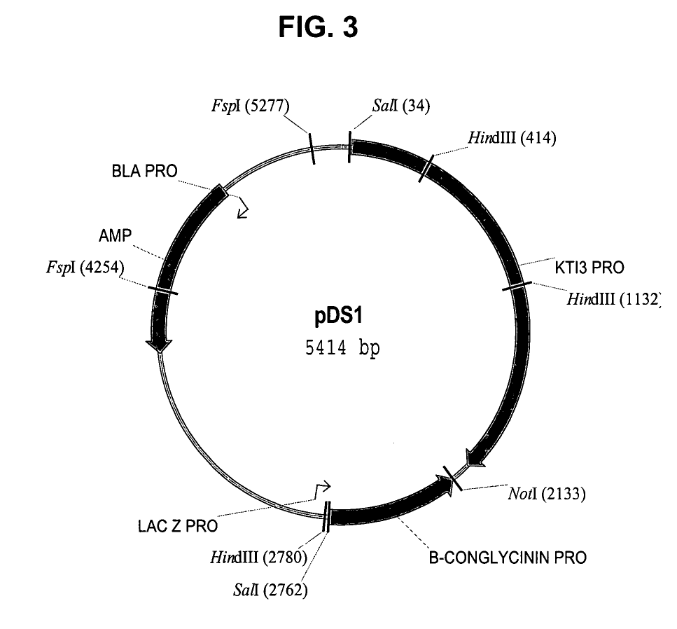

pKR57 (ATCC Accession No. PTA-6017) (FIG. 1) (4479 bp sequence; SEQ ID NO:1) was digested with Eco RI and Not I, run on a 0.8% Tris-Acetate-Ethylenediaminetetraacetic acid-agarose gel (TAE-agarose gel) and a 3144 bp fragment containing the β-conglycinin promoter, an origin of replication and a gene encoding ampicillin resistance was purified using the Qiagen gel extraction kit. pKR63 (ATCC Accession No. PTA-6018) (FIG. 2) (5010 bp sequence; SEQ ID NO:2) was digested with Eco RI and Not I, run on a 0.8% TAE-agarose gel and a 2270 bp fragment containing the KTi3 promoter was purified using the Qiagen gel extraction kit. The isolated fragments were ligated together and the ligation was transformed into E. coli and colonies were selected on ampicillin. Bacterial colonies were selected and grown overnight in LB media and appropriate antibiotic selection. DNA was isolated from the resulting culture using a Qiagen miniprep kit according to the manufacturer's protocol and then analyzed by restriction digest. The resulting plasmid was named pDS1 (FIG. 3) (5414 bp sequence; SEQ ID NO:3).

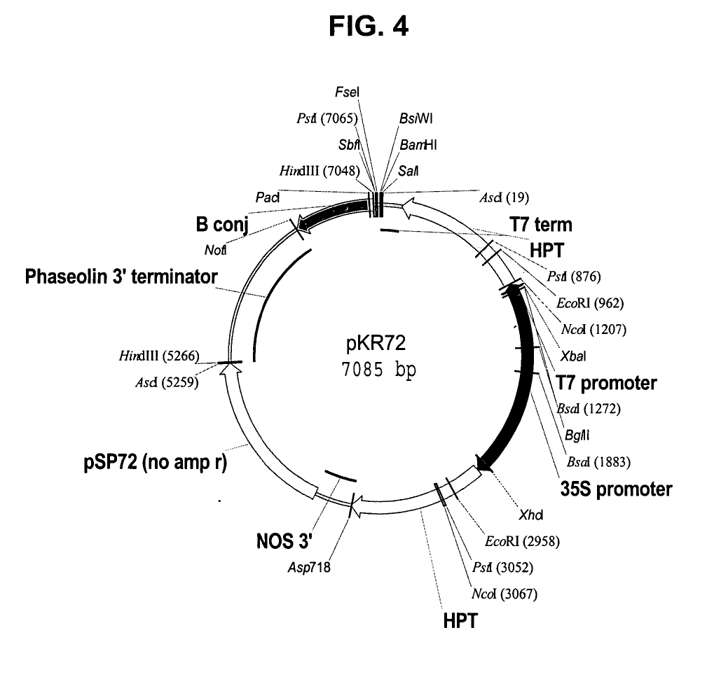

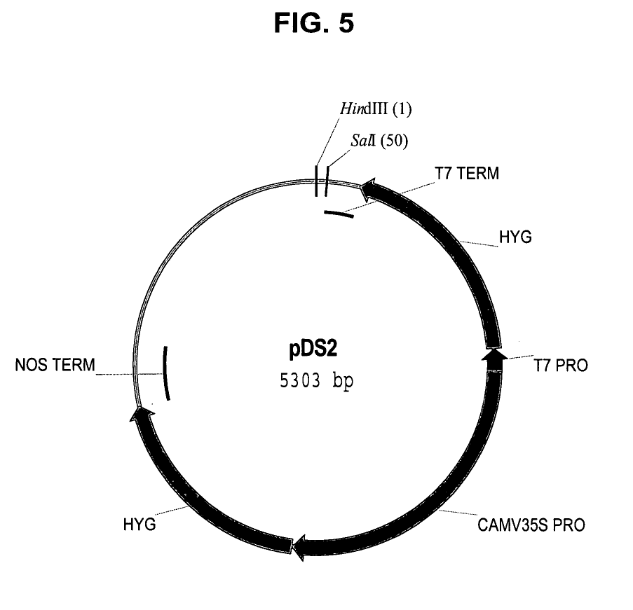

pKR72 (ATCC Accession No. PTA-6019) (FIG. 4) (7085 bp sequence; SEQ ID NO:4) was digested with Hind III, run on a 0.8% TAE-agarose gel and a 5303 bp fragment containing a gene that encodes resistance to hygromycin operably linked to a prokaryotic promoter and a gene that encodes resistance to hygromycin operably linked to a eukaryotic promoter were purified using the Qiagen gel extraction kit. The fragment was ligated to itself and the ligation was transformed into E. coli and colonies were selected on hygromycin. Bacterial colonies were selected and grown overnight in LB media and appropriate antibiotic selection. DNA was isolated from the resulting culture using a Qiagen miniprep kit according to the manufacturer's protocol and then analyzed by restriction digest. The resulting plasmid was named pDS2 (FIG. 5) (5303 bp sequence; SEQ ID NO:5).

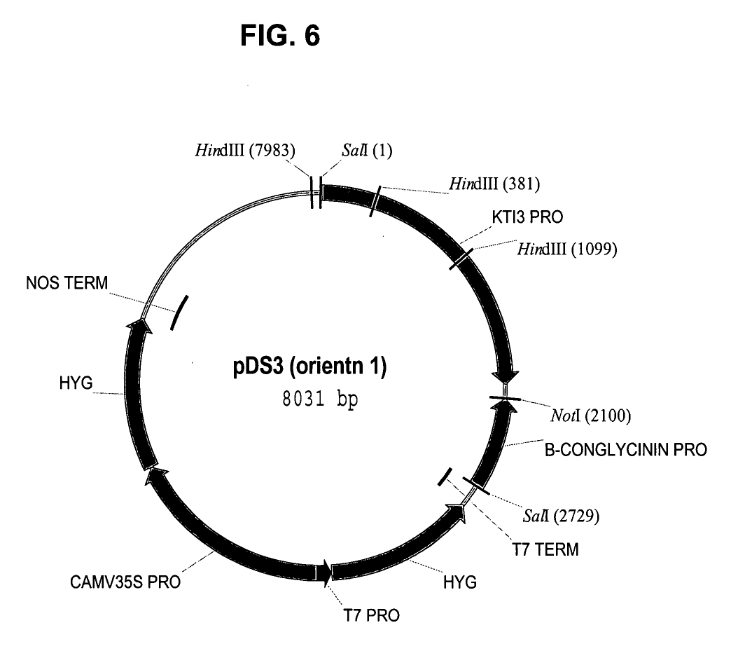

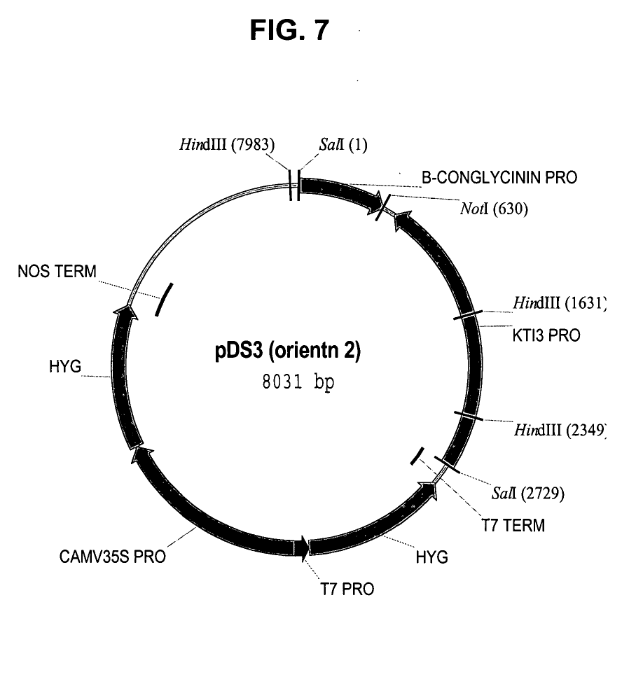

pDS2 was digested with Sal I and the ends were dephosphorylated with calf intestinal alkaline phosphatase (CIAP) according to the manufacture's instructions (Stratagene, San Diego, Calif.). pDS1 was digested with Sal I and Fsp I, run on a 0.8% TAE-agarose gel and a 2728 bp fragment containing the KTi3 promoter and the β-conglycinin promoter in opposite orientations was purified using the Qiagen gel extraction kit. The isolated fragments were ligated together and the ligation was transformed into E. coli and colonies were selected on hygromycin. Bacterial colonies were selected and grown overnight in LB media and appropriate antibiotic selection. DNA was isolated from the resulting culture using a Qiagen miniprep kit according to the manufacturer's protocol and then analyzed by restriction digest. The resulting plasmids were named pDS3 (orientation 1 and orientation 2) (FIG. 6 and FIG. 7, respectively) (8031 bp sequences; SEQ ID NO:6 and SEQ ID NO:7, respectively).

A 600 bp fragment was PCR amplified for soybean Fad2-1 using the following primers 5′-GMTTCGCGGCCGCTGAGTGATTGCTCACGAGT-3′ (SEQ ID NO:8) and 5′-GMTTCGCGGCCGCTTMTCTCTGTCCATAGTT-3′ (SEQ ID NO:9). The resulting fragment was cloned into a TA plasmid supplied with the TA cloning kit according to manufacture's instructions (Invitrogen, San Diego, Calif.). Bacterial colonies were selected and grown overnight in LB media and appropriate antibiotic selection. DNA was isolated from the resulting culture using a Qiagen miniprep kit according to the manufacturer's protocol and then analyzed by restriction digest. The resulting plasmid was digested with Not I and the fragment was cloned into Not I digested pDS3 (orientation 2) to form pDS5 (FIG. 8) (8642 bp sequence; SEQ ID NO:10).

### Example 2

### Transformation of Somatic Soybean (Glycine max) Embryo Cultures and Regeneration of Soybean Plants

The following example sets forth a protocol which can be used for transformation of soybean via particle bombardment of embryogenic tissue. Those skilled in the art will appreciate that a number of minor variations can be made to the protocol described below. Such transformed somatic embryos are also suitable for germination. The following protocol is also set forth in PCT Publication No. WO 02/00904, which published on Jan. 3, 2002.

Generic Stable Soybean Transformation Protocol:

Soybean embryogenic suspension cultures are maintained in 35 mL liquid media (SB55 or SBP6; see Table 1) on a rotary shaker, 150 rpm, at 28° C. with mixed fluorescent and incandescent lights on a 16:8 hour day/night schedule. Cultures are subcultured every four weeks by inoculating approximately 35 mg of tissue into 35 mL of liquid medium.

TABLE 1
Stock Solutions (g/L):

| MS Sulfate 100X Stock | | SB55 (per Liter, pH 5.7) |
|---|---|---|
| MgSO₄.7H₂O | 37.0 | 10 mL each MS stocks |
| MnSO₄.H₂O | 1.69 | 1 mL B5 vitamin stock |
| ZnSO₄.7H₂O | 0.86 | 0.8 g NH₄NO₃ |
| CuSO₄.5H₂O | 0.0025 | 3.033 g KNO₃ |
| MS Halides 100X Stock | | ‘ 1 mL 2,4-D (10 mg/mL stock) |
| CaCl₂.2H₂O | 44.0 | 60 g sucrose |
| KI | 0.083 | 0.667 g asparagine |
| CoCl₂.6H₂0 | 0.00125 | SBP6 |
| KH₂PO₄ | 17.0 | Same as SB55 except 0.5 mL 2,4-D |
| H₃BO₃ | 0.62 | SB103 (per Liter, pH 5.7) |
| Na₂MoO₄.2H₂O | 0.025 | 1X MS salts |
| MS FeEDTA 100X Stock | | 6% maltose |
| Na₂EDTA | 3.724 | 750 mg MgCl₂ |
| FeSO₄.7H₂O | 2.784 | 0.2% gelrite |

| | |
|---|---|
| B5 Vitamin Stock | SB71-1 (per Liter. pH 5.7) |
| 10 g m-inositol | 1X B5 salts |
| 100 mg nicotinic acid | 1 mL B5 vitamin stock |
| 100 mg pyridoxine.HCl | 3% sucrose |
| 1 g thiamine | 750 mg MgCl₂ |
| | 0.2% gelrite |

Soybean embryogenic suspension cultures are transformed with pTC3 by the method of particle gun bombardment (Klein et al., Nature 327:70 (1987)). A DuPont Biolistic PDS1000/HE instrument (helium retrofit) is used for these transformations.

To 50 mL of a 60 mg/mL 1 μm gold particle suspension is added (in order); 5 μL DNA (1 μg/1L), 20 μL spermidine (0.1 M), and 50 μL CaCl₂ (2.5 M). The particle preparation is agitated for 3 min, spun in a microfuge for 10 sec and the supernatant removed. The DNA-coated particles are then washed once in 400 μL 70% ethanol and resuspended in 40 μL of anhydrous ethanol. The DNA/particle suspension is sonicated three times for 1 sec each. Five μL of the DNA-coated gold particles are then loaded on each macro carrier disk. For selection, the plasmid contains a gene conferring resistance to hygromycin phosphotransferase (HYG) operably linked to an appropriate promoter. It is known by those skilled in the art that herbicide resistance can be used to select transformed plants in tissue culture, e.g., a mutated version of the acetolactate synthase (ALS) gene confers resistance to some sulfonylurea herbicides.

Approximately 300-400 mg of a four week old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1000 psi and the chamber is evacuated to a vacuum of 28 inches of mercury. The tissue is placed approximately 3.5 inches away from the retaining screen and bombarded three times. Following bombardment, the tissue is placed back into liquid and cultured as described above.

Eleven days post bombardment, the liquid media is exchanged with fresh SB55 containing 50 mg/mL hygromycin. The selective media is refreshed weekly. Seven weeks post bombardment, green, transformed tissue is observed growing from untransformed, necrotic embryogenic clusters. Isolated green tissue is removed and inoculated into individual flasks to generate new, clonally propagated, transformed embryogenic suspension cultures. Thus each new line is treated as an independent transformation event. These suspensions can then be maintained as suspensions of embryos maintained in an immature developmental stage or regenerated into whole plants by maturation and germination of individual somatic embryos.

Independent lines of transformed embryogenic clusters are removed from liquid culture and placed on a solid agar media (SB103) containing no hormones or antibiotics. Embryos are cultured for four weeks at 26° C. with mixed fluorescent and incandescent lights on a 16:8 hour day/night schedule. During this period, individual embryos are removed from the clusters and screened for alterations in their fatty acid compositions (Example 3).

It should be noted that any detectable phenotype, resulting from the co-suppression of a target nucleic acid fragment can be screened at this stage. The phenotype of transgenic soybean somatic embryos is predictive of seed phenotypes from resulting regenerated plants. This is further discussed in PCT Publication No. WO 02/00904, which published on Jan. 3, 2002. Detectable phenotypes include, but not be limited to, alterations in protein content, carbohydrate content, growth rate, viability, or the ability to develop normally into a soybean plant.

Furthermore, somatic embryos are also suitable for germination after eight weeks and can be removed from the maturation medium and dried in empty petri dishes for one to five days. The dried embryos can then be planted in SB71-1 medium where they will be allowed to germinate under the same lighting and germination conditions described above. Germinated embryos can be transferred to sterile soil and grown to maturity. Seeds can be harvested and analyzed for alteration in such things as their fatty acid compositions.

### Example 3

### Reduction of Expression of Fad2-1

The following example describes a reduction of the expression of Fad2-1 in soybean, Glycine max.

pDS5 (FIG. 8), as described in Example 1, was transformed into soybean embryogenic suspension cultures using a protocol as described in Example 2 above. Individual embryos were removed, at an appropriate time, from the clusters and screened for alterations in their fatty acid compositions. As was discussed above in Example 2, individual embryos behave in a manner similar to mature seeds and analysis of these embryos is predictive of the phenotype that will be found in mature seeds obtained from a transformed plant.

pDS5 contains the Fad2-1 gene situated between two convergent seed specific promoters, namely, the KTi3 promoter and the β-conglycinin promoter as was described in Example 1. Fad2-1 is a gene locus encoding a A¹² desaturase from soybean that introduces a double bond into the oleic acid chain to form a polyunsaturated fatty acid. Reduction in the expression of Fad2-1 results in the accumulation of oleic acid (18:1, or an 18 carbon fatty acid tail with a single double bond) and a corresponding decrease in polyunsaturated fatty acid content.

Control embryos (286 individuals) had an average 18:1 content of 9% with a standard deviation of 6.2% (actual range 4-22%). An oleic acid content of 25% or greater was chosen as a positive reduction in Fad2-1 which results in increased 18:1 that is more than two standard deviations from the mean, and higher than the highest control value seen. As mentioned above, another point to consider when analyzing transgenic plants with reduced expression due to co-suppression is chimerism. The analysis of the data set forth in this example takes into account the chimeric nature of tissue cultures. In the experiment described in PCT Application No. WO 02/00904, which published on Jan. 3, 2002, the positive event lines detected may have only contained a single embryo out of five with increased oleic acid content. If a line has little or no chimerism then all of its embryos will have a suppressed phenotype as opposed to exhibiting a wild type phenotype. Because of this, if a line has at least one embryo with an 18:1 content of 25% or more, it is counted as a positive event. Typically, five different embryos were analyzed for each event.

Twelve out of thirty-three (or 36%) lines analyzed showed increased levels of oleic acid, which is demonstrative of reduced gene expression (see Table 2).

TABLE 2
Positive Transformed Lines with Reduced Fad2-1 Expression

| | dsRNA |
|---|---|
| | Co-suppression* |
| Fad2-1 lines with | 12 out of 33 |
| >25% 18:1 content | |
| Percent total | 36% |

*Five different embryos were analyzed for each event. Any 18:1 >25% was considered dsRNA co-suppression (see Example 2).

Thus, this result shows that the method of the invention constitutes an efficient method for reducing gene expression.

### Example 4

### Construction of Galactinol Synthase Silencing Plasmids Driven by β-Conglycinin and KTi3

The following two examples describe a reduction of the expression of multiple galactinol synthase genes in soybean, Glycine max.

Raffinose saccharides are a group of D-galactose-containing oligosaccharide derivatives of sucrose that are widely distributed in plants. Raffinose saccharides are characterized by the following general formula: [O-β-D-galactopyranosyl-(1→6)ₙ-α-glucopyranosyl-(1→2)-β-D-fructofuranoside where n=0 through n=4 are known respectively as sucrose, raffinose, stachyose, verbascose and ajugose.

Although abundant in many species, raffinose saccharides are an obstacle to the efficient utilization of some economically important crop species. Raffinose saccharides are not digested directly by animals, primarily because alpha-galactosidase is not present in the intestinal mucosa (Gitzelmann et al., Pediatrics 36:231-236 (1965); Rutloff et al., Nahrung 11:39-46 (1967)). However, microflora in the lower gut are readily able to ferment the raffinose saccharides resulting in an acidification of the gut and production of carbon dioxide, methane and hydrogen gases (Murphy et al., J. Agr. Food. Chem. 20:813-817 (1972); Cristofaro et al., In Sugars in Nutrition; H. L. Sipple and K. W. McNutt, Eds. Academic Press: New York, Chap. 20, 1974; pp. 313-335; Reddy et al., J. Food Science 45:1161-1164 (1980)). The resulting flatulence can severely limit the use of leguminous plants in animal, particularly human, diets. It is unfortunate that the presence of raffinose saccharides restricts the use of legumes in human diets because many of these species are otherwise excellent sources of protein and soluble fiber. Varieties of edible beans free of raffinose saccharides would be more valuable for human and animal diets and would facilitate broader access to the desirable nutritional qualities of edible leguminous plants.

The biosynthesis of raffinose saccharides has been well-characterized (see Dey, P. M. In Biochemistry of Storage Carbohydrates in Green Plants; P. M. Dey and R. A. Dixon, Eds.; Academic Press: London, 1985, pp. 53-129). The committed reaction of raffinose saccharide biosynthesis involves the synthesis of galactinol from UDP-galactose and myo-inositol. The enzyme that catalyzes this reaction is galactinol synthase (inositol 1-alpha-galactosyltransferase; EC 2.4.1.123). Synthesis of raffinose and higher homologues in the raffinose saccharide family from sucrose is thought to be catalyzed by distinct galactosyltransferases (for example, raffinose synthase and stachyose synthase). Studies in many species suggest that galactinol synthase is the key enzyme controlling the flux of reduced carbon into the biosynthesis of raffinose saccharides (Handley et al., J. Amer. Soc. Hort. Sci. 108:600-605 (1983); Saravitz et al., Plant Physiol. 83:185-189 (1987)). Altering the activity of galactinol synthase, either as a result of overexpression or through gene silencing or antisense inhibition, would change the amount of raffinose saccharides produced in a given tissue.

Three genes encoding soybean galactinol synthases have been identified: galactinol synthase 1 (U.S. Pat. Nos. 5,773,699 and 5,648,210; Kerr et al, “Nucleotide Sequences of Galactinol Synthase from Zucchini and Soybean”), galactinol synthase 2 (PCT Publication No. WO 2001/077306, which published on Oct. 18, 2001; Allen et al., Plant Raffinose Saccharide Biosynthetic Enzymes) and galactinol synthase 3 (SEQ ID NO:11 of the instant invention). Since there are multiple genes encoding galactinol synthases (GAS), it is believed that suppression of more than one gene may be required to detect an effect on raffinose sugar levels.

Preparation of pJMS10:

Polynucleotide fragments encoding parts of the soybean galactinol synthase 1 (GAS1) (SEQ ID NO:12 which is identical to SEQ ID NO:6 of U.S. Pat. Nos. 5,773,699 and 5,648,210), galactinol synthase 2 (GAS2) in clone ses4d.pk0017.b8 (SEQ ID NO:13 which is identical to SEQ ID NO:3 of PCT Publication No. WO 2001/077306) and galactinol synthase 3 (GAS3) (SEQ ID NO:11) were amplified by standard PCR methods using Pfu Turbo DNA polymerase (Stratagene, La Jolla, Calif.) and the following primer sets. The GAS1 oligonucleotide primers were designed to add a Not I restriction endonuclease site at the 5′ end and a stopcodon (TGA) and an Xho I site to the 3′ end (SEQ ID NO:14 and SEQ ID NO:15, respectively). The DNA sequence comprising the 519 bp sequence from soybean GAS1 is shown in SEQ ID NO:16. The GAS2 oligonucleotide primers were designed to add an Xho I restriction endonuclease site at the 5′ end and a stopcodon (TAA) and a Pst I site to the 3′ end (SEQ ID NO:17 and SEQ ID NO:18, respectively). The DNA sequence comprising the 519 bp sequence from soybean GAS2 is shown in SEQ ID NO:19. The GAS3 oligonucleotide primers were designed to add a Pst I restriction endonuclease site at the 5′ end and a stopcodon (TAG) and a Not I site to the 3′ end (SEQ ID NO:10 and SEQ ID NO:21, respectively). The DNA sequence comprising the 519 bp sequence from soybean GAS3 is shown in SEQ ID NO:22.

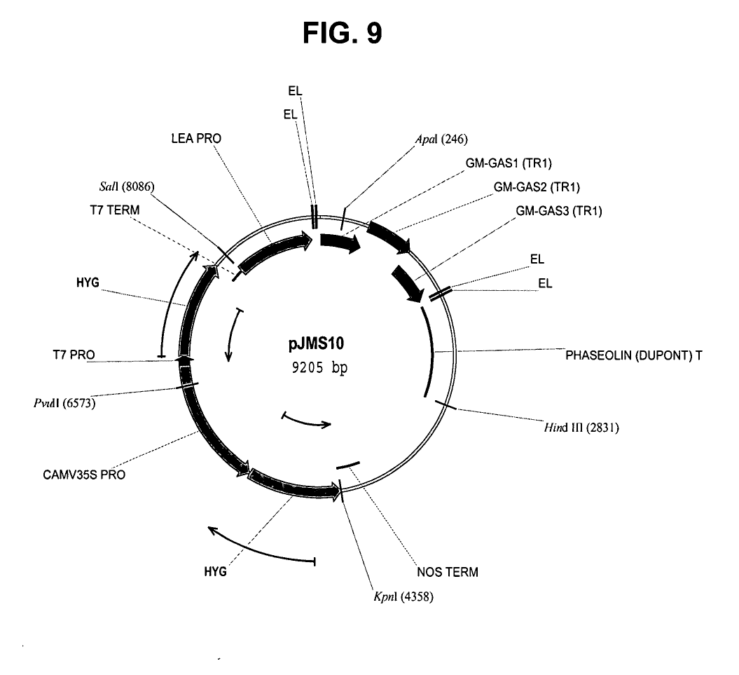

The polynucleotide products for GAS1 (SEQ ID NO:16), GAS2 (SEQ ID NO:19) and GAS3 (SEQ ID NO:22) obtained from the amplifications described above were digested with Not I, Xho I and Pst I and assembled into vector pJMS10 (FIG. 9) by the following steps. From plasmid KS123 (prepared according to U.S. Application No. 2004/0073975 A1, which published on Apr. 15, 2004) the Hind III cassette containing the beta-conglycinin promoter-phaseolin terminator was removed creating the plasmid KS120. To the unique BamH I site of plasmid KS120 a LEA promoter-phaseolin terminator was inserted as a BamH I fragment creating plasmid KS127. The LEA promoter (Lee et al., Plant Physiol. 100:2121-2122 (1992); GenBank Accession No. M97285) was amplified from genomic A2872 soybean DNA and a phaseolin 3′ end was added as described in U.S. Patent Publication No. 2003/0036197 A1. To KS127 an EL linker was added to a unique Not I site as described in U.S. Patent Publication No. 2003/0036197 A1, creating plasmid KS139. To KS139 an EL linker was added to a unique Not I site as described in U.S. Patent Publication No. 2003/0036197 A1, creating plasmid KS147. Plasmid KS147 also comprises nucleotides encoding hygromycin phosphotransferase (HPT) under the control of the T7 promoter and termination signals and the 35S promoter and Nos 3′. Next the isolated DNA fragments containing partial sequences of GAS1 (SEQ ID NO:16), GAS2 (SEQ ID NO:19) and GAS3 (SEQ ID NO:22) were inserted into the Not 1-digested plasmid KS147 to obtain plasmid pJMS10 (FIG. 9).

Preparation of SH60:

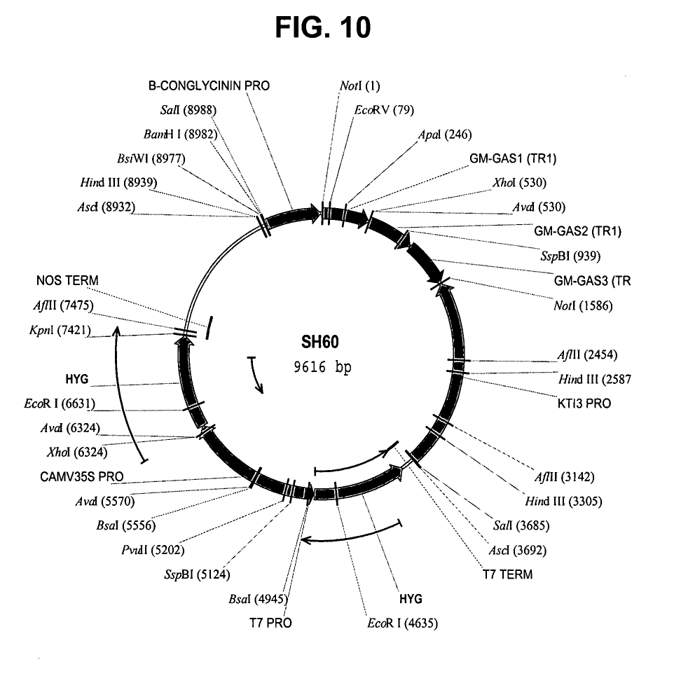

pJMS10 (FIG. 9) was digested with Not I, run on a 0.8% TAE-agarose gel and a 1585 bp DNA fragment (SEQ ID NO:25) comprising the partial sequences of GAS1 (SEQ ID NO:16), GAS2 (SEQ ID NO:19) and GAS3 (SEQ ID NO:22) was purified using the Qiagen gel extraction kit. pDS3 (orientation 2) described in Example 1 (FIG. 7) was digested with Not I, run on a 0.8% TAE-agarose gel and a 8031 bp DNA fragment (SEQ ID NO:69) was purified using the Qiagen gel extraction kit. The isolated fragments were ligated together and the ligation was transformed into E. coli and colonies were selected on hygromycin. Bacterial colonies were selected and grown overnight in LB media and appropriate antibiotic selection. DNA was isolated from the resulting culture using a Qiagen miniprep kit according to the manufacturer's protocol and then analyzed by restriction digest. The resulting plasmid was named SH60 (FIG. 10) (9616 bp sequence; SEQ ID NO:70).

### Example 5

### Reduction of Raffinose Family Oligosaccharides (RFOs) in Transenic Soybean Somatic Embryos

SH60, as described in Example 4, was transformed into soybean embryogenic suspension cultures using a similar protocol as described above in Example 2. Individual immature soybean embryos were dried-down (by transferring them into an empty small petri dish that was seated on top of a 10 cm petri dish containing some agar gel to allow slow dry down) to mimic the last stages of soybean seed development. Dried-down embryos are capable of producing plants when transferred to soil or soil-less media. Storage products produced by embryos at this stage are similar in composition to storage products produced by zygotic embryos at a similar stage of development and most importantly the storage product profile is predictive of plants derived from a somatic embryo line (PCT Publication No. WO 94/11516, which published on May 26, 1994). Raffinose Family Oligosaccharides (raffinose and stachyose) of transgenic somatic embryos containing the β-conglycinin/KTi3 driven (SH60) recombinant expression construct described in Example 4 was measured by thin layer chromatography (TLC). Somatic embryos were extracted with hexane then dried. The dried material was re-suspended in 80% methanol, incubated at room temperature for 1-2 hours, centrifuged, and 2 μL of the supernatant is spotted onto a TLC plate (Kieselgel 60 CF, from EM Scientific, Gibbstown, N.J.; Catalog No. 13749-6).

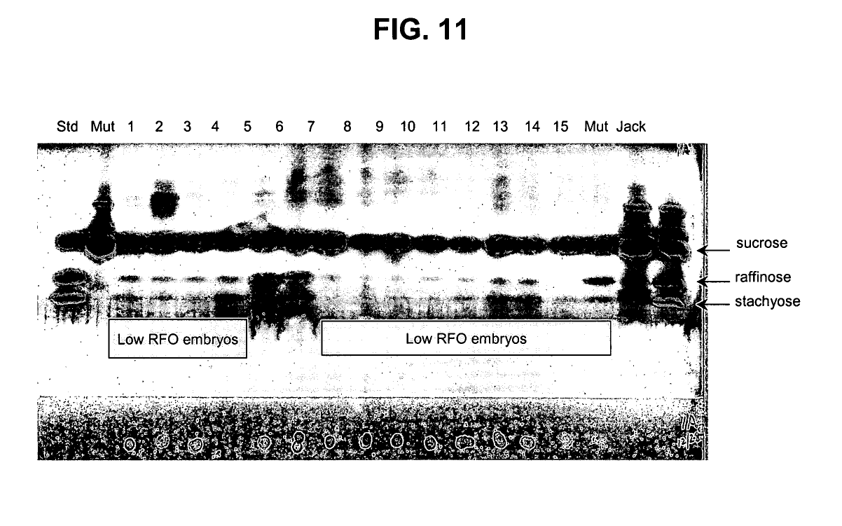

The TLC was run in ethylacetate:isopropanol:20% acetic acid (3:4:4) for 1-1.5 hours. The air dried plates were sprayed with 2% sulfuric acid and heated until the charred sugars were detected. As shown in FIG. 11 the embryos labeled “Low RFO embryos” show reduced levels of raffinose sugars (raffinose and stachyose) when compared to a to wild-type soybean. The RFO sugars (raffinose and stachyose) and sucrose from wild-type cultivar Jack are indicated with arrows. Mut is a mutant soybean line known to have very low levels of RFOs (less than 15% of wild-type). Numbers 1 to 15 represent samples from fifteen individual somatic embryos of “one” transgenic SH60 event. It is apparent that thirteen out of fifteen embryos have reduced RFO levels when compared to wild-type Jack.

Furthermore, five out of eleven lines analyzed (45%) showed reduced levels of RFOs, which is demonstrative of reduced galactinol synthase expression (see Table 3).

TABLE 3
Positive Transformed Lines with
Reduced Galactinol Synthase Expression

| | carbohydrate phenotype |
|---|---|
| GAS1GAS2GAS3 lines with wild-type RFO | 6 out of 11 |
| levels | |
| GAS1GAS2GAS3 lines with reduced RFO | 5 out of 11 |
| levels | |
| Percent gene silencing | 45% |

### Example 6

### Preparation of Plasmids

The ability to suppress expression of a number of target nucleic acid fragments was determined after transformation of somatic soybean embryos (see Example 7) with the following plasmids.

A. Construction of Plasmid DN10:

Plasmid pDN10 is an intermediate cloning vector comprising a bacterial origin of replication, bacterial and plant selectable marker gene expression cassettes, and a promoter and terminator separated by a unique Not I restriction endonuclease site. This plasmid was prepared by ligating a fragment comprising a plant selectable marker gene expression cassette and a cassette comprising a promoter and terminator separated by a unique Not I restriction endonuclease site to a fragment comprising the bacterial origin of replication and selectable marker gene. These two fragments were prepared as follows:

The first fragment has 6383 bp sequence, was obtained by Kpn I digestion of pKS231 (ATCC Accession No. PTA-6148), its nucleotide sequence is shown in SEQ ID NO:23, and contains the following two cassettes: 1) a plant selectable marker gene cassette, and 2) a cassette comprising a promoter and terminator separated by a unique Not I restriction endonuclease site. The plant selectable marker gene expression cassette comprises a 1.3-Kb DNA fragment that functions as the promoter for a soybean S-adenosylmethionine synthase (SAMS) gene directing expression of a mutant soybean acetolactate synthase (ALS) gene which is followed by the soybean ALS 3′ transcription terminator. The 1.3-Kb DNA fragment that functions as the promoter for a soybean SAMS gene has been previously described in PCT Publication No. WO 00/37662, published Jun. 29, 2000 (its entire contents are hereby incorporated by reference). The mutant soybean ALS gene encodes an enzyme that is resistant to inhibitors of ALS, such as sulfonylurea herbicides.

Mutant plant ALS genes encoding enzymes resistant to sulfonylurea herbicides are described in U.S. Pat. No. 5,013,659. One such mutant is the tobacco SURB-Hra gene, which encodes an herbicide-resistant ALS with two substitutions in the amino acid sequence of the protein. This tobacco herbicide-resistant ALS contains alanine instead of proline at position 191 in the conserved “subsequence B” and leucine instead of tryptophan at position 568 in the conserved “subsequence F” (U.S. Pat. No. 5,013,659; Lee et al., EMBO J. 7:1241-1248 (1988)).

The mutant soybean ALS gene was constructed using a polynucleotide sequence for a soybean ALS to which the two Hra-like mutations were introduced by site directed mutagenisis. Thus, this recombinant DNA fragment will translate to a soybean ALS having alanine instead of proline at position 183 and leucine instead of tryptophan at position 560. The deduced amino acid sequence of the mutant soybean ALS present in the mutant ALS gene is shown in SEQ ID NO:24. During construction of SAMS promoter-mutant ALS expression cassette, the coding region of the soybean ALS gene was extended at the 5′ end by five additional codons, resulting in five amino acids, added to the amino-terminus of the ALS protein (amino acids 1 through 5 of SEQ ID NO:24). These extra amino acids are adjacent to and presumably removed with the transit peptide during targeting of the mutant soybean ALS protein to the plastid.

The cassette comprising a promoter and terminator separated by a unique Not I restriction endonuclease site comprises the KTi3 promoter, a unique Not I restriction endonuclease site, and the KTi3 terminator region. This cassette comprises about 2088 nucleotides of the KTi3 promoter, a unique Not I restriction endonuclease site, and about 202 nucleotides of the KTi3 transcription terminator. The gene encoding KTi3 has been described (Jofuku, K. D. and Goldberg, R. B., Plant Cell 1:1079-1093 (1989)).

The second fragment, comprising the bacterial origin of replication and bacterial selectable marker gene was obtained by PCR amplification from plasmid pKS210 as follows. Plasmid pKS210 is derived from the commercially available cloning vector pSP72 (Promega, Madison, Wis.). To prepare plasmid pKS210 the beta lactamase coding region in vector pSP72 has been replaced by a hygromycin phosphotransferase (HPT) gene for use as a selectable marker in E. coli. The nucleotide sequence of plasmid pKS210 is shown in SEQ ID NO:26. A fragment of pKS210 comprising the bacterial origin of replication and the HPT gene was amplified by PCR using primers BM1 (SEQ ID NO:27) and BM2 (SEQ ID NO:28) and pKS210 as a template, and the Advantage High Fidelity polymerase (BD Biosciences, San Jose, Calif.) according to the manufacturer's instructions.

BM1:

SEQ ID NO:27-

5′-GCCGGGGTACCGGCGCGCCCGATCATCCGGATATAGTTCC-3′
BM2:

SEQ ID NO:28-

5′-GCCGGGGTACCGGCGCGCCGTTCTATAGTGTCACCTAATC-3′

A GeneAmp PCR System 9700 machine (Applied Biosystems, Foster City, Calif.) machine was used and the resulting 2600 bp fragment was gel purified using the Qiagen Gel Purification Kit, digested with Kpn I and treated with calf intestinal alkaline phosphatase.

The two Kpn I fragments described above were ligated together and transformed into E. coli. Bacterial colonies were selected and grown overnight in LB media and appropriate antibiotic selection. DNA was isolated from the resulting culture using a Qiagen Miniprep Kit according to the manufacturer's protocol and then analyzed by restriction digest. The resulting plasmid was named pDN10 and its nucleotide sequence is shown in SEQ ID NO:29.

B. Recombinant DNA Fragment KSFAD2-hybrid:

Recombinant DNA Fragment KSFAD2-hybrid contains an approximately 890 polynucleotide fragment comprising about 470 nucleotides from the soybean FAD2-2 gene and 420 nucleotides from the soybean FAD2-1 gene. The nucleotide sequence of recombinant DNA fragment KSFAD2-hybrid is shown in SEQ ID NO:30. Recombinant DNA Fragment KSFAD2-hybrid was constructed as follows.

An approximately 0.47 kb DNA fragment comprising a portion of the soybean FAD2-2 gene was obtained by PCR amplification using primers KS1 (the nucleotide sequence of which is shown in SEQ ID NO:31) and KS2 (the nucleotide sequence of which is shown in SEQ ID NO:32) and using genomic DNA purified from leaves of Glycine max cv. Jack as a template.

| | |
|---|---|
| KS1: | |
| 5′-GCGGCCGCCGGTCCTCTCTCTTTCCGTG-3′ | SEQ ID NO:31- |
| KS2: | |
| 5′-TAGAGAGAGTAAGTCCTGCAAGTACTCCTG-3′ | SEQ ID NO:32- |

An approximately 0.42 kb DNA fragment comprising a portion of the soybean FAD2-1 gene was obtained by PCR amplification using primers KS3 (the nucleotide sequence of which is shown in SEQ ID NO:33) and KS4 (the nucleotide sequence of which is shown in SEQ ID NO:34) and using genomic DNA purified from leaves of Glycine max cv. Jack as a template.

| | |
|---|---|
| KS3: | |
| 5′-CAGGAGTACTTGCAGGACTTACTCTCTCTA-3′ | SEQ ID NO:33- |
| KS4: | |
| 5′-GCGGCCGGCCCCTTCTCGGATGTTCCTTC-3′ | SEQ ID NO:34- |

The 0.47 kb fragment comprising a portion of the soybean FAD2-2 gene and the 0.42 kb fragment comprising a portion of the soybean FAD2-1 gene were gel purified using GeneClean (Qbiogene, Irvine, Calif.), mixed, and used as template for PCR amplification with KS1 and KS4 as primers (SEQ ID NO:31 and SEQ ID NO:34, respectively) to yield an approximately 890 bp fragment that was cloned into the commercially available plasmid pGEM-T Easy (Promega, Madison, Wis.) to create a plasmid comprising recombinant DNA Fragment KSFAD2-hybrid.

C. Construction of Recombinant DNA Fragment 1028:

Recombinant DNA fragment 1028 was constructed to provide additional sequence similarity to the LOX1 and LOX2 genes in order to more efficiently suppress expression of all three soybean seed lipoxygenase genes. Recombinant DNA fragment 1028 (the 4351 bp sequence of which is shown in SEQ ID NO:35) comprises the following in 5′ to 3′ orientation:
a) about 2088 nucleotides of the KTi3 promoter;
b) 74-nucleotide synthetic sequence;
c) a unique Eco RI restriction endonuclease site containing a 1364-nucleotide DNA fragment from the soybean LOX3 gene and a 523-nucleotide DNA fragment from the soybean LOX2 gene;
d) an inverted repeat of the nucleotides in b); and
e) about 202 nucleotides of the KTi3 transcription terminator.

The nucleotide synthetic sequences in b) and d) promote formation of a stem in a stem-loop structure where the nucleotide fragment of c) forms the loop. This stem-loop structure has been shown to result in suppression of the gene having similarity to the nucleotide fragment forming the loop as described in PCT Publication WO 02/00904, published Jan. 3, 2002.

cDNAs encoding entire soybean seed LOX2 or LOX3 were identified by BLAST analysis and comparison to known sequences in cDNA libraries that are part of a proprietary collection of EST sequences. A cDNA encoding an entire soybean LOX2 was identified as clone se4.pk0007.e7 (SEQ ID NO:47) and was found in a library prepared from soybean embryos nineteen days after flowering. A cDNA encoding an entire soybean LOX3 was identified as clone sgs1c.pk002.g4 (SEQ ID NO:48) and was found in a library prepared from soybean cotyledons seven days after germination.

To construct recombinant DNA fragment 1028, a seed-specific gene expression-silencing cassette was obtained from vector pKS133 and modified. Vector pKS133 has been described in PCT Publication WO 02/00904, published Jan. 3, 2002, and is derived from the commercially available vector pSP72 (Promega, Madison, Wis.). To generate recombinant DNA fragment 1028 the seed-specific gene expression-silencing cassette from pKS133 was modified by replacing the unique Not I site with a unique Eco RI site and inserting into this unique site a polynucleotide from a soybean seed lipoxygenase 3 (LOX3) gene. The unique Eco RI site was generated by inserting into the Not I site of pKS133, by DNA ligation, a self-annealing oligonucleotide linker. A 2226 nucleotide DNA fragment from the soybean seed lipoxygenase 3 was obtained by digesting with Eco RI the cDNA insert in clone sgs1c.pk002.g4 (SEQ ID NO:48), and was then inserted into the Eco RI site of the gene expression-silencing cassette. Next, an 862-nucleotide fragment from the soybean LOX3 gene in this recombinant DNA plasmid was removed by digestion with Pst I and Sph I. This fragment was replaced with a 523 nucleotide soybean LOX2 DNA fragment obtained by digestion of clone se4.pk0007.e7 (SEQ ID NO:47) with Pst I and Sph I. This 523 nucleotide soybean LOX2 DNA fragment contains 3 regions with 32 or more contiguous nucleotides that are identical between soybean LOX1 and soybean LOX2 genes; the longest common sequence is 50 contiguous nucleotides (shown in SEQ ID NO:36).

D. Construction of Recombinant Plasmid DS8:

Plasmid DS1 (Example 1—FIG. 3) was digested with Sal1 and Not I and the resulting fragments were electrophoresed on a TAE agarose gel. The resulting 629 bp band comprising the 1-conglycinin promoter (Chen et al., Dev. Genet. 10:112-122 (1989)) was purified using a Qiagen Gel Purification Kit.

Plasmid DN10 (Example 6A) was digested with Not I and Xho I and the resulting fragments were electrophoresed on a TAE agarose gel. The resulting 8627 bp band was purified using a Qiagen Gel Purification Kit.

The above two purified fragments were ligated together and transformed into E. coli. DNA fragments with Sal1 and Xho I sites have compatible overhangs and can be ligated together. Bacterial colonies were selected and grown overnight in LB media and appropriate antibiotic selection. DNA was isolated from the resulting culture using a Qiagen Miniprep Kit according to the manufacturer's protocol and then analyzed by restriction digest. The resulting 9256 bp plasmid was named pDS8 and its nucleotide sequence is shown in SEQ ID NO:37.

E. Construction of Recombinant Plasmid PHP21676:

Recombinant DNA plasmid PHP21676 contains sequences designed to silence expression of seed lipoxygenases (LOX), the FAD2-1 and FAD2-2 genes, and the FAD3 gene. The nucleotide sequence of plasmid PHP21676 is shown in SEQ ID NO:38. Plasmid PHP21676 contains an approximately 3414 polynucleotide fragment comprising in 5′ to 3′ orientation about 470 nucleotides from the soybean FAD2-2 gene, 420 nucleotides from the soybean FAD2-1 gene, 643 nucleotides from the soybean FAD3 gene and about 1880 nucleotides from the soybean LOX3 and LOX2 genes inserted between Not I restriction endonuclease sites. The sequence of the approximately 3414 polynucleotide fragment is shown in SEQ ID NO:39 and was constructed by PCR amplification as follows.

An approximately 0.9 kb DNA fragment, comprising a portion of the soybean FAD2-2 gene and a portion of the soybean FAD2-1 gene, was obtained by PCR amplification using primers BM3 (the nucleotide sequence of which is shown in SEQ ID NO:40) and BM4 (the nucleotide sequence of which is shown in SEQ ID NO:41) and using as template recombinant DNA fragment KSFAD2-hybrid described in Example 6A above.

| | |
|---|---|
| BM3: | |
| 5′-GCGGCCGCCGGTCCTCTCTCTTTCCGTG-3′ | SEQ ID NO:40- |
| BM4: | |
| 5′-TAAACGGTGGAGGAGCCCTTCTCGGATGTTC-3′ | SEQ ID NO:41- |

An approximately 0.65 kb DNA fragment, comprising a portion of a FAD3 gene, was obtained by PCR amplification using primers BM5 (the nucleotide sequence of which is shown in SEQ ID NO:42) and BM6 (the nucleotide sequence of which is shown in SEQ ID NO:43) and using plasmid pXF1 (ATCC Accession No. 68874) as template. Plasmid pXF1 comprises a polynucleotide sequence encoding a soybean delta-15 desaturase (FAD3) and is described in U.S. Pat. No. 5,952,544 which issued on Sep. 14, 1999. Plasmid pXF1 was deposited with the American Type Culture Collection (ATCC) of Rockville, Md. on Dec. 3, 1991 under the provisions of the Budapest Treaty, and bears ATCC Accession Number 68874.

BM5:

SEQ ID NO:42-

5′-GAACATCCGAGAAGGGCTCCTCCACCGTTTAAG-3′
BM6:

SEQ ID NO:43-

5′-GCGGCCGCCCATAGAGCTTGAGCACTAG-3′

The approximately 0.9 kb fragment, comprising a portion of the soybean FAD2-2 gene and a portion of the soybean FAD2-1 gene, and the approximately 0.65 kb fragment, comprising a portion of a FAD3 gene, were mixed and used as template for a PCR amplification with BM3 and BM6 (SEQ ID NO:40 and SEQ ID NO:43, respectively) as primers to yield an approximately 1533 bp fragment that was cloned into the commercially available plasmid pCR2.1 using the TOPO TA Cloning Kit (Invitrogen) to form plasmid Taste24/pCR-TOPO.

An approximately 1.5 kb DNA fragment, comprising a portion of the soybean FAD2-2 gene, a portion of the soybean FAD2-1 gene, and a portion of the soybean FAD3 gene, was obtained by PCR amplification using primers BM3 (the nucleotide sequence of which is shown in SEQ ID NO:40) and BM7 (the nucleotide sequence of which is shown in SEQ ID NO:44) and using plasmid Taste24/pCR-TOPO as a template.

BM3:

SEQ ID NO:40-

5′-GCGGCCGCCGGTCCTCTCTCTTTCCGTG-3′
BM7:

SEQ ID NO:44-

5′-TAAAATGCTCCAGGAATTCCATAGAGCTTGAGCAC-3′

An approximately 1.9 kb DNA fragment, comprising portions of the LOX2 and LOX3 genes, was obtained by PCR amplification using primers BM8 (the nucleotide sequence of which is shown in SEQ ID NO:45) and BM9 (the nucleotide sequence of which is shown in SEQ ID NO:46) and using recombinant DNA fragment 1028 as template. Recombinant DNA fragment 1028 is described in Example 6C, above.

BM8:

SEQ ID NO:45-

5′-GCGGCCGCCCTCTGAAAGTTAATCCTTCC-3′
BM9:

SEQ ID NO:46-

5′-GCTCAAGCTCTATGGAATTCCTGGAGCATTTTATATC-3′

The approximately 1.5 kb fragment, comprising a portion of the FAD2-2 gene, a portion of the FAD2-1 gene, and a portion of the FAD3 gene, was mixed with the approximately 1.9 kb fragment, comprising portions of the LOX2 and LOX3 genes, and used as template for a PCR amplification with BM3 and BM8 as primers (SEQ ID NO:40 and SEQ ID NO:45, respectively) to yield an approximately 3414 bp fragment that was cloned into the commercially available plasmid pCR2.1 using the TOPO TA Cloning Kit (Invitrogen).

After digestion with Not I the approximately 3414 bp fragment having the nucleotide sequence shown in SEQ ID NO:39 was ligated into the Not I site of plasmid pKS210, described in Example 6A above, to form plasmid PHP21676 shown in SEQ ID NO:38.

F. Construction of Recombinant Fragments PHP22905A and PHP22972A:

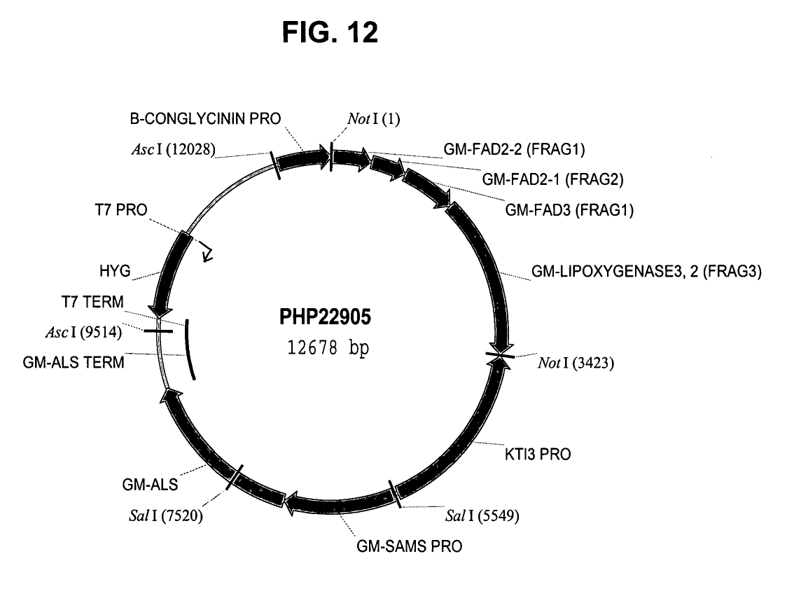

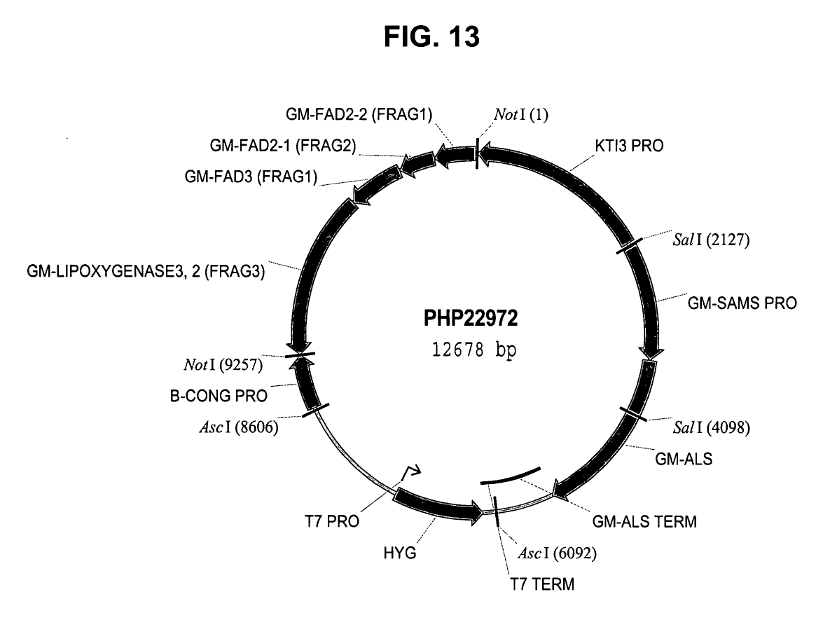

After digestion of plasmid PHP21676 (SEQ ID NO:48) with Not I the approximately 3414 bp fragment having the nucleotide sequence shown in SEQ ID NO:39 was ligated into the Not I site of plasmid pDS8 (SEQ ID NO:37), described in Example 6D above, to form the plasmids PHP22905 (SEQ ID NO:49) and PHP22972 (SEQ ID NO:50). These plasmids differ in their orientation of the 3414 bp fragment in respect to the convergent promoters (FIG. 12 and FIG. 13).

For use in plant transformation experiments the 9874 bp recombinant DNA fragments PHP22905A (SEQ ID NO:51) and PHP22972A (SEQ ID NO:52) were removed from their cloning plasmid using restriction endonuclease Asc I and were separated from the remaining plasmid DNAs by agarose gel electrophoresis.

G. Construction of Recombinant Fragment PHP23466A:

A search for nucleic acids from Glycine max (soybean) that encode polypeptides with similarity to the amino acid sequence of the Euphorbia lagascae CYP726A1 (SEQ ID NO:53; NCBI Accession No. ML62063.1) was carried out using a tBLASTn search against a proprietary database containing contigs assembled from ESTs and/or full-insert sequences of soybean cDNAs from both public and private sources. Contigs are nucleotide sequences assembled from constituent nucleotide sequences that share common or overlapping regions of sequence identity. The tBLASTn algorithm is used to search an amino acid query against a nucleotide database that is translated in all six reading frames.

This tBLASTn analysis resulted in several contigs encoding polypeptides with significant homology to the Euphorbia lagascae CYP726A1. The nucleotide sequence of the entire cDNA insert in clone sfl1.pk0045.g7 (shown in SEQ ID NO:54) is part of one such contig, and the polynucleotide sequence of sfl1.pk0045.g7 encompasses the complete contig.

Clone sfl1.pk0045.g7 is derived from a library prepared from soybean (Glycine max L., Wye) immature flowers. The deduced amino acid sequence obtained from translating nucleotides 22 through 1548 of SEQ ID NO:54 is shown in SEQ ID NO:55. The sfl1.pk0045.g7 full insert sequence polynucleotide has been shown to produce 1-octen-3-ol when expressed in yeast in the presence of linoleic acid.

An approximately 1100 bp fragment, comprising a portion of the P450-EPOX gene was amplified with primers BM10 (the nucleotide sequence of which is shown in SEQ ID NO:56) and BM11 (the nucleotide sequence of which is shown in SEQ ID NO:57) using cDNA sfl1.pk0045.g7 (SEQ ID NO:55) as a template.

BM10:

SEQ ID NO:56-

5′-GCGGCCGCATGGCTCTATTATTCTTCTACTTTTTG-3′
BM11:

SEQ ID NO:57-

5′-CTTGATATAAAATGCTCCAGGAATTCAACCTCAAGGTCTCTTTCAC-
3′

An approximately 1880 bp fragment, comprising a portion of the lipoxygenase 2 and lipoxygenase 3 genes was amplified with primers BM12 (the nucleotide sequence of which is shown in SEQ ID NO:58) and BM13 (the nucleotide sequence of which is shown in SEQ ID NO:59) using PHP21676 (SEQ ID NO:48) (Example 6E above) as a template.

BM12:

SEQ ID NO:58-

5′-GTGAAAGAGACCTTGAGGTTGAATTCCTGGAGCATTTTATATCAAG-
3′
BM13:

SEQ ID NO:59-

5′-GCGGCCGCCCTCTGAAAGTTAATCCTTCC-3′

The approximately 1.1 kb fragment, comprising a portion of the P450-EPOX gene, was mixed with the approximately 1.9 kb fragment, comprising portions of the LOX2 and LOX3 genes, and used as template for a PCR amplification with BM10 and BM13 as primers (SEQ ID NO:56 and SEQ ID NO:59, respectively) to yield an approximately 2993 bp fragment with SEQ ID NO:60 that was cloned into the commercially available plasmid pCR2.1 using the TOPO TA Cloning Kit (Invitrogen) to form the plasmid with the SEQ ID NO:61.

After digestion of the plasmid with the SEQ ID NO:61 with Not I the approximately 2985 bp fragment having the nucleotide sequence shown in SEQ ID NO:60 was ligated into the Not I site of plasmid pDS, described in Example 6D above, to form recombinant plasmid PHP23466 with SEQ ID NO:62.

For use in plant transformation experiments the approximately 9735 bp recombinant DNA fragment PHP23466A (SEQ ID NO:63) was removed from its cloning plasmid (SEQ ID NO:62) using restriction endonuclease Asc I and was separated from the remaining plasmid DNA by agarose gel electrophoresis.

H. Construction of Recombinant Fragment PHP23465A:

An approximately 1100 bp fragment, comprising a portion of the P450-EPOX gene was amplified with primers BM10 (the nucleotide sequence of which is shown in SEQ ID NO:56) and BM14 (the nucleotide sequence of which is shown in SEQ ID NO:63) using cDNA sfl1.pk0045.g7 (SEQ ID NO:55) as a template. This approximately 1100 bp fragment was cloned into the commercially available plasmid pCR2.1 using the TOPO TA Cloning Kit (Invitrogen) and the sequence of this plasmid is shown in SEQ ID NO:64.

BM10:

SEQ ID NO:56-

5′-GCGGCCGCATGGCTCTATTATTCTTCTACTTTTTG-3′
BM14:

SEQ ID NO:63-

5′-CTCGAGCAACCTCAAGGTCTCTTTCACAATTAG-3′

An approximately 3420 bp fragment described in 6E above (SEQ ID NO:39) comprising a portion of the FAD2-2 gene, a portion of the FAD2-1 gene, a portion of the FAD3 gene, and portions of the LOX2 and LOX3 genes was amplified with primers BM15 (the nucleotide sequence of which is shown in SEQ ID NO:65) and BM13 (the nucleotide sequence of which is shown in SEQ ID NO:59) using PHP21676 (SEQ ID NO:48) as template. This approximately 3420 bp fragment was cloned into the commercially available plasmid pCR2.1 using the TOPO TA Cloning Kit (Invitrogen) and the sequence of this plasmid is shown in SEQ ID NO:66.

BM15:

SEQ ID NO:65-

5′-CTCGAGCGGTCCTCTCTCTTTCCGTGGCATGGC-3′
BM13:

SEQ ID NO:59-

5′-GCGGCCGCCCTCTGAAAGTTAATCCTTCC-3′

The plasmids shown in SEQ ID NO:64 and SEQ ID NO:66 were subject to restriction digestion with the enzymes Xho I and Not I and subjected to gel electrophoresis. From SEQ ID NO:64 an approximately 1100 bp fragment was purified and from SEQ ID NO:66 an approximately 3414 bp fragment was purified using the Qiagen Gel Purification Kit. Plasmid DS8 (SEQ ID NO:37) was subjected to restriction digest with Not I and treated with calf intestinal alkaline phosphatase. The three fragments were ligated together and transformed into E. coli. Bacterial colonies were selected and grown overnight in LB media and appropriate antibiotic selection. DNA was isolated from the resulting culture using a Qiagen Miniprep Kit according to the manufacturer's protocol and then analyzed by restriction digest. The resulting plasmid was named PHP23465 and its nucleotide sequence is shown in SEQ ID NO:67.

For use in plant transformation experiments the approximately 11274 bp recombinant DNA fragment PHP23465A (SEQ ID NO:68) was removed from its cloning plasmid (SEQ ID NO:67) using restriction endonuclease Asc I and was separated from the remaining plasmid DNA by agarose gel electrophoresis.

### Example 7

### Transformation of Somatic Soybean (Glycine max) Embryo Cultures and Regeneration of Soybean Plants

Soybean embryogenic suspension cultures were transformed by the method of particle gun bombardment using a similar protocol as described above in Example 2 and procedures known in the art (Klein et al., Nature (London) 327:70-73 (1987); U.S. Pat. No. 4,945,050; Hazel et al., Plant Cell. Rep. 17:765-772 (1998); Samoylov et al., In Vitro Cell Dev. Biol.—Plant 34:8-13 (1998)). In particle gun bombardment procedures it is possible to use either purified 1) entire plasmid DNA or, 2) DNA fragments containing only the recombinant DNA expression cassette(s) of interest.

For transformation of PHP22905A (SEQ ID NO:51), PHP22972A (SEQ ID NO:52), PHP23466A (SEQ ID NO:63), and PHP23465A (SEQ ID NO:68), the recombinant DNA fragments were isolated from the entire plasmid by Asc I digestion and gel electrophoresis before being used for bombardment. For every eight bombardment transformations, 30 microliters of solution were prepared with 3 mg of 0.6 mm gold particles and 1 to 90 picograms (pg) of DNA fragment per base pair of DNA fragment. The DNA/particle suspension was sonicated three times for one second each. Five microliters of the DNA-coated gold particles were then loaded on each macro carrier disk.

Stock tissue for these transformation experiments were obtained by initiation from soybean immature seeds. Secondary embryos were excised from explants after 6 to 8 weeks on culture initiation medium. The initiation medium was an agar-solidified modified MS (Murashige and Skoog, Physiol. Plant 15:473-497 (1962)) medium supplemented with vitamins, 2,4-D and glucose. Secondary embryos were placed in flasks in liquid culture maintenance medium and maintained for seven to nine days on a gyratory shaker at 26+/−2° C. under ˜80 μEm⁻²s⁻¹ light intensity. The culture maintenance medium was a modified MS medium supplemented with vitamins, 2,4-D, sucrose and asparagine. Prior to bombardment, clumps of tissue were removed from the flasks and moved to an empty 60×15 mm petri dish for bombardment. Tissue was dried by blotting on Whatman #2 filter paper. Approximately 100-200 mg of tissue corresponding to 10-20 clumps (1-5 mm in size each) were used per plate of bombarded tissue.

After bombardment, tissue from each bombarded plate was divided and placed into two flasks of liquid culture maintenance medium per plate of bombarded tissue. Seven days post bombardment, the liquid medium in each flask was replaced with fresh culture maintenance medium supplemented with 100 ng/mL selective agent (selection medium). For selection of transformed soybean cells the selective agent used was a sulfonylurea (SU) compound with the chemical name, 2-chloro-N-((4-methoxy-6 methy-1,3,5-triazine-2-yl)aminocarbonyl) benzenesulfonamide (common names: DPX-W4189 and chlorsulfuron). Chlorsulfuron is the active ingredient in the DuPont sulfonylurea herbicide, GLEAN®. The selection medium containing SU was replaced every week for six to eight weeks. After the six to eigth week selection period, islands of green, transformed tissue were observed growing from untransformed, necrotic embryogenic clusters. These putative transgenic events were isolated and kept in media with SU at 100 ng/mL for another two to six weeks with media changes every one to two weeks to generate new, clonally propagated, transformed embryogenic suspension cultures. Embryos spent a total of around eight to twelve weeks in SU.

Suspension cultures were subcultured and maintained as clusters of immature embryos and will be regenerated into whole plants by maturation and germination of individual somatic embryos. Individual somatic embryos were also harvested for analysis as described below in Example 8. Previous work has shown that analysis of embryos is predictive of the phenotype obtained in seeds from regenerated plants (PCT Publication No. WO 94/11516, which published on May 26, 1994).

### Example 8

### Assays for Indication of Suppression of Genes

A. Assay For Fatty Acid Composition:

In order to determine whether the fatty acid composition was altered, which would indicate suppression of the fatty acid desaturase gene expression, the relative amounts of the fatty acids, palmitic, stearic, oleic, linoleic and linolenic, in soybean somatic embryos was determined as follows. Fatty acid methyl esters were prepared from single, mature, somatic soybean embryos by transesterification. One embryo was placed in a vial containing 50 μL of trimethylsulfonium hydroxide and incubated for thirty minutes at room temperature while shaking. After the thirty minutes 0.5 mL of hexane was added, the sample was mixed and allowed to settle for fifteen to thirty minutes to allow the fatty acids to partition into the hexane phase. Fatty acid methyl esters (5 μL from hexane layer) were injected, separated, and quantified using a Hewlett-Packard 6890 Gas Chromatograph fitted with an Omegawax 320 fused silica capillary column (Supelco Inc., Catalog #24152). The oven temperature was programmed to hold at 220° C. for 2.7 minutes, increase to 240° C. at 20° C. per minute, and then hold for an additional 2.3 minutes. Carrier gas was supplied with a Whatman hydrogen generator. Retention times were compared to those for methyl esters of commercially available standards (Nu-Chek Prep, Inc. Catalog #U-99-A).

An increase in oleic acid is indicative of suppression of fatty acid desaturase gene(s).

B. Assay for Soybean LOX1:

Lipoxygenase activity was determined using a spectrophotometric assay where sodium linoleate is hydroperoxidated increasing the 234 nm absorbance of the sample. When measuring LOX1 activity in soybeans (Glycine max cv. Jack) the absorbance at 234 nm increases in one to three minutes to about 0.5 or 0.6 OD234 nm min⁻¹.

Sodium linoleate substrate was prepared from linoleic acid as follows. Seventy mg of linoleic acid and 70 mg of Tween 20 were weighed out into a 50 mL tube and homogenized in 4 mL sterile filtered double deionized (ddi) water. About 0.55 mL of 0.5 N sodium hydroxide was added in order to obtain a clear solution. Sterile filtered double distilled water was added to bring the solution up to 25 mL total volume. The solution was divided in 2 mL aliquots, which were stored at −20° C. under nitrogen gas. The final stock concentration of sodium linoleate was 10 mM.

To prepare extract from soybean somatic embryos, three-week-old somatic soybean embryos were individually ground in 500 μL of 2 mM sodium taurodeoxycholate in a microtiter plate (96 deep-well microtiter plates with a 1.2-2 mL working volume per well) using one 4 mm or 5/32″ steel grinding ball per embryo. The embryos were ground with two 30-45 second cycles at 1500 strokes/min using a Geno/Grinder™ (SPEX CertiPrep, Metuchen, N.J.). The microtiter plates were then centrifuged using a Sorvall Super T21 centrifuge at 500 to 700 rpm for five minutes to remove cellular debris.

To measure lipoxygenase activity in soybean somatic embryos 10 μL of the extract from above was decanted from each well and transferred to a 96-well standard UV grade microtiter plate suitable for a microtiter plate reader. To each well 100 μL of 0.2 mM sodium linoleate (18:2) in 0.1 M sodium borate, pH 9.0 was added and the increase in absorbance at 234 nm was monitored for three to five minutes using a microtiter plate reader SpectraMax 190 (Molecular Devices Corp., Sunnyvale, Calif.).

The assay described in this Example was specific for the detection of LOX1. No lipoxygenase activity was observed when this assay was performed on seeds of a soybean mutant known to lack LOX1, and which contains lipoxygenase isozymes LOX2 and LOX3. In contrast, lipoxygenase activity was observed when this assay was performed on seeds of soybean mutants known to contain LOX1 and lack either LOX2 or LOX3. Thus, the somatic embryo LOX1 assay provides a useful test for selection of transformation events likely to yield LOX1 null seeds.

None of the recombinant DNA fragments designed to suppress soybean seed lipoxygenases contained more than 50 contiguous nucleotides from the LOX1 gene. Therefore, it was expected that seeds that lacked LOX1 enzyme activity would also lack LOX2 and LOX3 activities, as one or both of these were present in the recombinant DNA fragments.

### Example 9

### Assays for Indication of Suppression of Genes

Embryos transformed with various recombinant fragments were assayed for the absence of lipoxygenase 1 (LOX1) activity. Only embryos that did not show lipoxygenase activity were further assayed for fatty acid content. Results are shown in Table 4.

TABLE 4
Analysis of Embryos

| Recombinant | Total Embryos | Suppressed | | P450- |
|---|---|---|---|---|
| Fragment | Analyzed | LOX | High Oleic | EPOX |
| PHP22905A | 32 | 11 (34%) | 2 (18%) | N/A |
| (SEQ ID NO:51) | | | | |
| PHP22972A | 72 | 23 (32%) | 12 (52%) | N/A |
| (SEQ ID NO:52) | | | | |
| PHP23466A | 143 | 24 (17%) | N/A | N/D |
| (SEQ ID NO:63) | | | | |
| PHP23465A | 115 | 24 (21%) | 9 (38%) | N/D |
| (SEQ ID NO:68) | | | | |

N/A = not applicable
N/D = not determined

The results show that 18% (2/11) of LOX suppressed had high oleic for PHP22905A, 52% (12/23) of LOX suppressed had high oleic for PHP22972A and 38% (9/24) of LOX suppressed had high oleic for PHP23465A.

This analysis shows that a plant organ stably transformed with a recombinant construct comprising at least one nucleic acid fragment of interest inserted between two convergent promoters will have a reduction in expression of the target nucleic acid fragments of interest.

What is claimed is:

1. A method for reducing expression of at least one target nucleic acid fragment in a plant or plant organ, the method comprising:
(a) stably transforming a plant cell with a recombinant construct comprising at least one isolated nucleic acid fragment of interest situated between a first and second promoter wherein
(i) the first and second promoters may be the same or different;
(ii) the first and second promoters have similar spatial and temporal activity; and
(iii) the first and second promoters are convergent;
further wherein the recombinant construct is stably integrated into the genome of the plant cell;
(b) regenerating a transformed plant or plant organ from the plant cell of (a); and
(c) evaluating the transformed plant or plant organ for reduced expression of the target nucleic acid fragment when compared to a nontransformed plant or plant organ.

2. The method of claim 1 wherein the first and second promoters are selected from the group consisting of: constitutive promoter, inducible promoter, tissue-type specific promoter, developmental type-specific promoter, cell type-specific promoter and seed-specific promoter.

3. The method of claim 1 wherein the first and second promoters are seed-specific promoters selected from the group consisting of: beta-conglycinin promoter, Kunitz soybean trypsin inhibitor promoter, napin promoter, beta-phaseolin promoter, oleosin promoter, albumin promoter, zein promoter, Bce4 promoter and legumin B4 promoter.

4. The method of claim 1 wherein the plant cell is selected from the group consisting of: soybean, corn, alfalfa, canola, sorghum, sunflower, wheat, rice oat, cotton, rye, sorghum, sugarcane, tomato, tobacco, millet, flax, potato, barley, Arabidopsis, bean, pea, rape, safflower, asparagus, beet, broccoli, cabbage, carrot, cauliflower, celery, cucumber, eggplant, lettuce, onion, pepper, potato, pumpkin, radish, spinach, squash, taro, tomato, zucchini, almond, apple, apricot, banana, blackberry, blueberry, cacao, cherry, coconut, cranberry, date, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut and watermelon.

5. A recombinant construct for reducing expression of at least one target nucleic acid fragment in a plant cell or plant organ, said construct comprising at least one isolated nucleic acid fragment of interest situated between a first and second promoter wherein
(i) the first and second promoters may be the same or different;
(ii) the first and second promoters have similar spatial and temporal activity; and
(iii) the first and second promoters are convergent;
further wherein the recombinant construct is stably integrated into the genome of the plant cell.

6. The recombinant construct of claim 5 wherein the first and second promoters are selected from the group consisting of: constitutive promoter, inducible promoter, tissue-type specific promoter, developmental type-specific promoter, cell type-specific promoter and seed-specific promoter.

7. The recombinant construct of claim 5 wherein the first and second promoters are seed-specific promoters selected from the group consisting of: beta-conglycinin promoter, Kunitz soybean trypsin inhibitor promoter, napin promoter, beta-phaseolin promoter, oleosin promoter, albumin promoter, zein promoter, Bce4 promoter and legumin B4 promoter.

8. The recombinant construct of claim 5 wherein the plant cell is selected from the group of: soybean, corn, alfalfa, canola, sorghum, sunflower, wheat, oat, cotton, rye, sorghum, sugarcane, tomato, tobacco, millet, flax, potato, barley, Arabidopsis, bean, pea, rape, safflower, asparagus, beet, broccoli, cabbage, carrot, cauliflower, celery, cucumber, eggplant, lettuce, onion, pepper, potato, pumpkin, radish, spinach, squash, taro, tomato, zucchini, almond, apple, apricot, banana, blackberry, blueberry, cacao, cherry, coconut, cranberry, date, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut and watermelon.

9. A transgenic plant or plant organ stably transformed with the recombinant construct of claim 5.

10. A recombinant construct comprising a sequence selected from the group consisting of: SEQ ID NO:10, SEQ ID NO:51, SEQ ID NO:52, SEQ ID NO:63, SEQ ID NO:68 and SEQ ID NO:70.

11. A method for reducing expression of at least one target nucleic acid fragment in a plant or plant organ, the method comprising:
(a) stably transforming a plant cell with a recombinant construct comprising a sequence selected from the group consisting of: SEQ ID NO:10, SEQ ID NO:51, SEQ ID NO:52, SEQ ID NO:63, SEQ ID NO:68 and SEQ ID NO:70,
wherein the recombinant construct is stably integrated into the genome of the plant cell;
(b) regenerating a transformed plant or plant organ from the plant cell of (a); and
(c) evaluating the transformed plant or plant organ for reduced expression of the target nucleic acid fragment when compared to a nontransformed plant or plant organ.


SEQUENCE LISTING (70 sequences)

SEQ ID NO: 1 - DNA, 4479 bp, Artificial
  plasmid pKR57
ctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagacgtacgttgaaacatccctgaagtgtctcattttattttatttattctttgctgataaaaaaataaaataaaagaagctaagcacacggtcaaccattgctctactgctaaaagggttatgtgtagtgttttactgcataaattatgcagcaaacaagacaactcaaattaaaaaatttcctttgcttgtttttttgttgtctctgacttgactttcttgtggaagttggttgtataaggattgggacaccattgtccttcttaatttaattttattctttgctgataaaaaaaaaaatttcatatagtgttaaataataatttgttaaataaccaaaaagtcaaatatgtttactctcgtttaaataattgagattcgtccagcaaggctaaacgattgtatagatttatgacaatatttacttttttatagataaatgttatattataataaatttatatacatatattatatgttatttattattattttaaatccttcaatattttatcaaaccaactcataattttttttttatctgtaagaagcaataaaattaaatagacccactttaaggatgatccaacctttatacagagtaagagagttcaaatagtaccctttcatatacatatcaactaaaatattagaaatatcatggatcaaaccttataaagacattaaataagtggataagtataatatataaatgggtagtatataatatataaatggatacaaacttctctctttataattgttatgtctccttaacatcctaatataatacataagtgggtaatatataatatataaatggagacaaacttcttccattataattgttatgtcttcttaacacttatgtctcgttcacaatgctaaggttagaattgtttagaaagtcttatagtacacatttgtttttgtactatttgaagcattccataagccgtcacgattcagatgatttataataataagaggaaatttatcatagaacaataaggtgcatagatagagtgttaatatatcataacatcctttgtttattcatagaagaagtgagatggagctcagttattatactgttacatggtcggatacaatattccatgctctccatgagctcttacacctacatgcattttagttcatacttgcggccgcagtatatcttaaattctttaatacggtgtactaggatattgaactggttcttgatgatgaaaacctgggccgagattgcagctatttatagtcataggtcttgttaacatgcatggacatttggccacggggtggcatgcagtttgacgggtgttgaaataaacaaaaatgaggtggcggaagagaatacgagtttgaggttgggttagaaacaacaaatgtgagggctcatgatgggttgagttggtgaatgttttgggctgctcgattgacacctttgtgagtacgtgttgttgtgcatggcttttggggtccagtttttttttcttgacgcggcgatcctgatcagctagtggataagtgatgtccactgtgtgtgattgcgtttttgtttgaattttatgaacttagacattgctatgcaaaggatactctcattgtgttttgtcttcttttgttccttggctttttcttatgatccaagagactagtcagtgttgtggcattcgagactaccaagattaattatgatgggggaaggataagtaactgattagtacggactgttaccaaattaattaataagcggcaaatgaagggcatggatcggccggcct

SEQ ID NO: 2 - DNA, 5010 bp, Artificial
  plasmid pKR63
ggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggtcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccggccggcctaaagggcggatcccccgggctgca

SEQ ID NO: 3 - DNA, 5414 bp, Artificial
  plasmid pDS1
aattcgagctcggtacccggggatcctctagagtcgacctgcaggtcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgcagtatatcttaaattctttaatacggtgtactaggatattgaactggttcttgatgatgaaaacctgggccgagattgcagctatttatagtcataggtcttgttaacatgcatggacatttggccacggggtggcatgcagtttgacgggtgttgaaataaacaaaaatgaggtggcggaagagaatacgagtttgaggttgggttagaaacaacaaatgtgagggctcatgatgggttgagttggtgaatgttttgggctgctcgattgacacctttgtgagtacgtgttgttgtgcatggcttttggggtccagtttttttttcttgacgcggcgatcctgatcagctagtggataagtgatgtccactgtgtgtgattgcgtttttgtttgaattttatgaacttagacattgctatgcaaaggatactctcattgtgttttgtcttcttttgttccttggctttttcttatgatccaagagactagtcagtgttgtggcattcgagactaccaagattaattatgatgggggaaggataagtaactgattagtacggactgttaccaaattaattaataagcggcaaatgaagggcatggatcggccggcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtg

SEQ ID NO: 4 - DNA, 7085 bp, Artificial
  plasmid pKR72
gtacggatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatcgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttgttgaaacatccctgaagtgtctcattttattttatttattctttgctgataaaaaaataaaataaaagaagctaagcacacggtcaaccattgctctactgctaaaagggttatgtgtagtgttttactgcataaattatgcagcaaacaagacaactcaaattaaaaaatttcctttgcttgtttttttgttgtctctgacttgactttcttgtggaagttggttgtataaggattgggacaccattgtccttcttaatttaattttattctttgctgataaaaaaaaaaatttcatatagtgttaaataataatttgttaaataaccaaaaagtcaaatatgtttactctcgtttaaataattgagattcgtccagcaaggctaaacgattgtatagatttatgacaatatttacttttttatagataaatgttatattataataaatttatatacatatattatatgttatttattattattttaaatccttcaatattttatcaaaccaactcataattttttttttatctgtaagaagcaataaaattaaatagacccactttaaggatgatccaacctttatacagagtaagagagttcaaatagtaccctttcatatacatatcaactaaaatattagaaatatcatggatcaaaccttataaagacattaaataagtggataagtataatatataaatgggtagtatataatatataaatggatacaaacttctctctttataattgttatgtctccttaacatcctaatataatacataagtgggtaatatataatatataaatggagacaaacttcttccattataattgttatgtcttcttaacacttatgtctcgttcacaatgctaaggttagaattgtttagaaagtcttatagtacacatttgtttttgtactatttgaagcattccataagccgtcacgattcagatgatttataataataagaggaaatttatcatagaacaataaggtgcatagatagagtgttaatatatcataacatcctttgtttattcatagaagaagtgagatggagctcagttattatactgttacatggtcggatacaatattccatgctctccatgagctcttacacctacatgcattttagttcatacttgcggccgcagtatatcttaaattctttaatacggtgtactaggatattgaactggttcttgatgatgaaaacctgggccgagattgcagctatttatagtcataggtcttgttaacatgcatggacatttggccacggggtggcatgcagtttgacgggtgttgaaataaacaaaaatgaggtggcggaagagaatacgagtttgaggttgggttagaaacaacaaatgtgagggctcatgatgggttgagttggtgaatgttttgggctgctcgattgacacctttgtgagtacgtgttgttgtgcatggcttttggggtccagtttttttttcttgacgcggcgatcctgatcagctagtggataagtgatgtccactgtgtgtgattgcgtttttgtttgaattttatgaacttagacattgctatgcaaaggatactctcattgtgttttgtcttcttttgttccttggctttttcttatgatccaagagactagtcagtgttgtggcattcgagactaccaagattaattatgatgggggaaggataagtaactgattagtacggactgttaccaaattaattaataagcggcaaatgaagggcatggatcaaaagcttggatctcctgcaggatctggccggccggatctc

SEQ ID NO: 5 - DNA, 5303 bp, Artificial
  plasmid pDS2
agcttggatctcctgcaggatctggccggccggatctcgtacggatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatcgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgcca

SEQ ID NO: 6 - DNA, 8031 bp, Artificial
  plasmid pDS3 (orientation 1)
tcgacctgcaggtcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgcagtatatcttaaattctttaatacggtgtactaggatattgaactggttcttgatgatgaaaacctgggccgagattgcagctatttatagtcataggtcttgttaacatgcatggacatttggccacggggtggcatgcagtttgacgggtgttgaaataaacaaaaatgaggtggcggaagagaatacgagtttgaggttgggttagaaacaacaaatgtgagggctcatgatgggttgagttggtgaatgttttgggctgctcgattgacacctttgtgagtacgtgttgttgtgcatggcttttggggtccagtttttttttcttgacgcggcgatcctgatcagctagtggataagtgatgtccactgtgtgtgattgcgtttttgtttgaattttatgaacttagacattgctatgcaaaggatactctcattgtgttttgtcttcttttgttccttggctttttcttatgatccaagagactagtcagtgttgtggcattcgagactaccaagattaattatgatgggggaaggataagtaactgattagtacggactgttaccaaattaattaataagcggcaaatgaagggcatggatcggccggcctctagagtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatcgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatctcctgcaggatctggccggccggatctcgtacggatccg

SEQ ID NO: 7 - DNA, 8031 bp, Artificial
  plasmid pDS3 (orientation 2)
tcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaatgtgttattaacccttctcttcgaggacctgcaggtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatcgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatctcctgcaggatctggccggccggatctcgtacggatccg

SEQ ID NO: 8 - DNA, 33 bp, Artificial
  primer
gaattcgcggccgctgagtgattgctcacgagt

SEQ ID NO: 9 - DNA, 33 bp, Artificial
  primer
gaattcgcggccgcttaatctctgtccatagtt

SEQ ID NO: 10 - DNA, 8642 bp, Artificial
  plasmid pDS5
ggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaatgtgttattaacccttctcttcgaggacctgcaggtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatcgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatctcctgcaggatctggccggccggatctcgtacggatccgtcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgctgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaagc

SEQ ID NO: 11 - DNA, 1151 bp, Glycine max
ctaagctctcttttagtcttactcacaaacacttttttcactgcttccattacgaacatatatttattatatggctcctgaacttgtccccaccgttgtgaaatccagtgctgcgttcacgaaacccgcgacccttccaaggcgtgcctacgtgacattcctcgccggaaacggtgactacgtgaaaggggtggttggcctcgccaaagggttgcgaaaggtgaaaaccgcgtacccgttggtggtggctgtcctccccgatgtgccggaggagcaccgtaagatcctggagtctcagggctgcatcgttcgcgagatcgaacccgtttacccacccgaaaaccaaacccagtttgccatggcttattacgtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaaggaagagaatatgcagagggaggacataaagatgctggtgaagaaatggtgggatatctacaatgatgcttcgcttgactacaagccattgatgaatgcaagtgaagctccagcagcggatggtgttgacattgaacaattcgtgcaggctctatcagaggttggtcatgttcaatatgtcaccgcgccttcagcagcttaattaagagggcacattcaaatcacgacaaaaaacaaccaagtgaaaaaaaaaaaaaaaaaaa

SEQ ID NO: 12 - DNA, 1406 bp, Glycine max
gtttgttttcaaagtgtgttttgtttcccaaatcctactcttgtgaccacaacccttcctcctctttcttttgaaacctctttttttctattccccaaccaaacaagcaaacgctactcactcatcatcactgagatcatggctcctaatatcaccactgtcaaaaccaccatcaccgacgctcaagccaaggtcgccaccgatcatggtcgtgcctacgtcaccttcctcgccggaaacggtgactatgtgaaaggtgtcgttggcttggcaaaaggtctgagaaaagtgaagagcatgtaccctctggtggttgcagtgctacccgatgttccccaagatcaccgcaacattctcacctcccaaggttgcattgttagagagattgagcccgtgtaccccccagagaatcaaacccagtttgccatggcatattacgtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaaggaggagaatatggagagagaagatatcaagatgttagtgaaaaagtggtgggatatatatgaggatgagactttggactacaacaatccactcaatgtggataagttcactgcggcacttatggaggttggtgaagtcaagttcgtccgtgccccatctgctgcttaagagtgtctttggaaatcaagtgtgatccaagtacatgtacaaagtcatacatcattacattaacttttatgtatttctaaaagtcatacatcattacattaagttttatgtatttctaaagtcttaagacttaagaggaccttttttatkkkkcccgcttttctttttttctttttccaattctgtcattgtaaagsrgagaataccgtatccttaattttataaatggatatgaattttatttgtactaaagggggggccggtaccaattcgcctatagt

SEQ ID NO: 13 - DNA, 1350 bp, Glycine max
gcacgagaaacaaccaacctcttcagtgatctttgattagtactaagctaaaccatttcttattccctcaaaatcaaaacctttttctttctagctatttcccttttcaaatcatgccacctaacatcaccaccgttgttgccaatgtcaccaccgagcaattacccaaggctcgtggaggaagtgggcgtgccttcgtgacctttcttgctgggaacggtgattacgtaaagggtgtcgtgggtttggccaaaggactgagaaaggccaaaagcatgtaccctttggtggttgctgtgttaccagatgttcctgaagaacatcgtgagattctcaaatcccaaggttgcattgtcagggagattgaacctgtgtaccctcctgagaaccagacccagttcgccatggcctattatgtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaaggaagagaacatggatagggaagatatcaagatgcttgtgaagaagtggtgggacatatatgaagatgagacactggactacaataacaactctgtcaacgtggaacgtttcacatcggcactattggatgctgggggctttcagtttgtgccagcaccttctgctgcctaatatgcttattatttacagctacaaattaatgttaattaacgacaaagtatatgtattgttatttgctttttttcgtttttgggtcttatatatgaaggaacaacgtctatggttttaatttggatgaccttcttgtatacaaagccacatgtgatctcatacagcttttgattattattaagaaattagaggaccttttattatgagtcctttacttaaaaaaaaaaaaaaaaaaaaaaaaaaa

SEQ ID NO: 14 - DNA, 36 bp, Artificial
  primer
aagcttgcggccgcgtcatcaactattccaagctac

SEQ ID NO: 15 - DNA, 36 bp, Artificial
  primer
aagcttctcgagtcacttcccagtgtacctccaagg

SEQ ID NO: 16 - DNA, 519 bp, Glycine max
gtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaag

SEQ ID NO: 17 - DNA, 34 bp, Artificial
  primer
aagcttctcgaggtcatcaattactccaagctac

SEQ ID NO: 18 - DNA, 44 bp, Artificial
  primer
aagcttgcggccgcctgcagttacttcccagtgaacctccaagg

SEQ ID NO: 19 - DNA, 519 bp, Glycine max
gtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaag

SEQ ID NO: 20 - DNA, 42 bp, Artificial
  primer
aagcttgcggccgcctgcaggtcatcaactactccaagctcc

SEQ ID NO: 21 - DNA, 37 bp, Artificial
  primer
aagcttgcggccgctacttccccgtatatctccatgg

SEQ ID NO: 22 - DNA, 519 bp, Glycine max
gtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaag

SEQ ID NO: 23 - DNA, 6383 bp, Artificial
  sequence obtained from Kpn I digestion of pKS231
cgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 24 - protein, 656 aa, Glycine max
MPHNTMAATASRTTRFSSSSSHPTFPKRITRSTLPLSHQTLTKPNHALKIKCSISKPPTAAPFTKEAPTTEPFVSRFASGEPRKGADILVEALERQGVTTVFAYPGGASMEIHQALTRSAAIRNVLPRHEQGGVFAAEGYARSSGLPGVCIATSGPGATNLVSGLADALMDSVPVVAITGQVARRMIGTDAFQETPIVEVSRSITKHNYLILDVDDIPRVVAEAFFVATSGRPGPVLIDIPKDVQQQLAVPNWDEPVNLPGYLARLPRPPAEAQLEHIVRLIMEAQKPVLYVGGGSLNSSAELRRFVELTGIPVASTLMGLGTFPIGDEYSLQMLGMHGTVYANYAVDNSDLLLAFGVRFDDRVTGKLEAFASRAKIVHIDIDSAEIGKNKQAHVSVCADLKLALKGINMILEEKGVEGKFDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQLEDRFYKSNRAHTYLGDPSSESEIFPNMLKFADACGIPAARVTKKEELRAAIQRMLDTPGPYLLDVIVPHQEHVLPMIPSNGSFKDVITEGDGRTRY

SEQ ID NO: 25 - DNA, 1585 bp, Artificial
  need to add something
ggccgcgtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaagtgactcgaggtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaagtaactgcaggtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaagtagc

SEQ ID NO: 26 - DNA, 8966 bp, Artificial
  plasmid pKS210
gatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcg

SEQ ID NO: 27 - DNA, 40 bp, Artificial
  primer
gccggggtaccggcgcgcccgatcatccggatatagttcc

SEQ ID NO: 28 - DNA, 40 bp, Artificial
  primer
gccggggtaccggcgcgccgttctatagtgtcacctaatc

SEQ ID NO: 29 - DNA, 8911 bp, Artificial
  plasmid pDN10
cggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 30 - DNA, 890 bp, Artificial
  recombinant DNA fragment KSFAD2-hybrid
cggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaaggg

SEQ ID NO: 31 - DNA, 28 bp, Artificial
  primer
gcggccgccggtcctctctctttccgtg

SEQ ID NO: 32 - DNA, 30 bp, Artificial
  primer
tagagagagtaagtcctgcaagtactcctg

SEQ ID NO: 33 - DNA, 30 bp, Artificial
  primer
caggagtacttgcaggacttactctctcta

SEQ ID NO: 34 - DNA, 29 bp, Artificial
  primer
gcggccggccccttctcggatgttccttc

SEQ ID NO: 35 - DNA, 4351 bp, Artificial
  recombinant DNA fragment 1028
cgcgccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggaattcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 36 - DNA, 50 bp, Glycine max
cttctagaaggttgcttcctgagaaaggcaccccagaatatgaagaaatg

SEQ ID NO: 37 - DNA, 9256 bp, Artificial
  plasmid pDS8
cggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 38 - DNA, 12388 bp, Artificial
  plasmid PHP21676
ggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgttcccacccagtgagagaaaaggaatagcaatatcaacactgtgttgggctaccatgttttctctgcttatctatctctcattcataactagtccacttctagtgctcaagctctatggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagggc

SEQ ID NO: 39 - DNA, 3414 bp, Artificial
  sequence constructed from PCR amplification
cggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgttcccacccagtgagagaaaaggaatagcaatatcaacactgtgttgggctaccatgttttctctgcttatctatctctcattcataactagtccacttctagtgctcaagctctatggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagg

SEQ ID NO: 40 - DNA, 28 bp, Artificial
  primer
gcggccgccggtcctctctctttccgtg

SEQ ID NO: 41 - DNA, 31 bp, Artificial
  primer
taaacggtggaggagcccttctcggatgttc

SEQ ID NO: 42 - DNA, 33 bp, Artificial
  primer
gaacatccgagaagggctcctccaccgtttaag

SEQ ID NO: 43 - DNA, 28 bp, Artificial
  primer
gcggccgcccatagagcttgagcactag

SEQ ID NO: 44 - DNA, 35 bp, Artificial
  primer
taaaatgctccaggaattccatagagcttgagcac

SEQ ID NO: 45 - DNA, 29 bp, Artificial
  primer
gcggccgccctctgaaagttaatccttcc

SEQ ID NO: 46 - DNA, 37 bp, Artificial
  primer
gctcaagctctatggaattcctggagcattttatatc

SEQ ID NO: 47 - DNA, 2917 bp, Glycine max
ttcggcacgagcttgttctttaattttctgattttagcaaattaaaatagtagtgttattggtaggttttggcaaagatgttttcagttccaggggtgtcgggaatcctgaacagaggaggagggcataagataaaagggacagtggtgttgatgaggaagaatgtgttggacttcaacagcgtggctgatcttactaaaggaaatgttgggggactcataggcaccggcctcaacgttgttggctcaacacttgataacctcactgctttcttgggccgaagtgtcgccctacagctcattagtgctaccaaacctcttgcaaatggaaaaggaaaagttggaaaggatacgttcttggaagggattattgtgtcgttaccaactttgggagcaggggagtctgcattcaatattcagtttgaatgggacgaaagcatgggaatccccggtgcgttttacataaagaactacatgcaagttgagttttacctcaagagtttgactcttgaagacgttccaaaccaaggaaccattcgctttgtttgcaactcatgggtttataacactaaactttacaaaagcgtgcgcattttctttgccaaccatacatatgttccaagtgagacaccagcagcacttgtggggtacagagaagaagaattgaagaatttaagaggagatggaaaaggagagcgcaaagaacatgataggatctatgattatgatgtctacaatgatttgggcaatccagatcacggtgaaaattttgctcgcccaattcttggagggtctagcactcatccctaccctcgtaggggaagaactggtcgatatccaacaaggaaagatcagaattctgagaagccaggcgaagtttatgttccaagagatgaaaattttggtcacttgaagtcctcggattttcttgcatatggaataaaatctttatctcaatatgtcttaccggcgttcgaatctgttttcgatttgaacttcacgccaaatgagtgtgatagcttccaagatgtgcgtgacctccacgaaggtggaattaagcttcctacagaagtaattagcacaattatgcccttaccggtggtcaaagaactctttcgtacagatggtgaacaagtcctcaagtttccaccacctcatgtcattcaagtgagtaagtctgcatggatgaccgatgaagaatttgcaagagagatggttgcgggtgtaaatccatgcgtaattcgtggtcttcaagagtttcctccaaaaagcaatctggatcctacaatctatggtgagcaaaccagtaagataacagcagatgcccttgatctagatggatacacagtggacgaggcactagcaagtcggaggttatttatgttagattaccatgatgtattcatgccatatataaggcggataaatcagacatatgccaaggcttatgcgacaaggactatcctttttctgagagaaaatggaaccttaaagccagtggccatcgaattaagtttgccacatcctgctggggacctgtcaggtgctgtcagtcaagtcatcttacctgcaaaagaaggtgttgaaagtacaatttggctactggccaaagcttatgtggtcgtaaatgactcttgctatcatcaactcatgagccattggttaaatactcatgcggtgattgagccattcatcatagcaacaaaccgacaccttagtgctcttcacccaatttataagcttctaactcctcactaccgtgacaccatgaacatcaacgcacttgctaggcaatctctcattaatgctgatggcataatagagaaatcttttttgccctcaaagcattccgttgagatgtcttcagcggtttataagaattgggttttcactgatcaagcactacctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcttctgatgaggtctaccttggccaaagggacaacccacattggacctctgactcaaaagcattacaagcctttcaaaaatttggaaacaagctcaaagaaattgaggaaaaacttgcaaggaagaacaatgatcaaagtctctccaatcgacttgggccggttcaactgccatacactttgctccatcctaacagtgaggaagggttgacttgcagggggattcctaatagcatctctatctaagggagcatgtggtctactttaattacagtactgtacctaccttccaataaaaaagatgcaaggctagagatccaataaatcttgcatcctatctaatgtttcaattatcttgtgttttaatgctgtaatggagcctacaatttgcaaattaattaagagcaccgagcactattagtttgcgtttaaaaaaaaaaaaaaaaaaaaaaaaaaaaaaaaaaaaaaaaaaaaaaaaaaaaaaa

SEQ ID NO: 48 - DNA, 2794 bp, Glycine max
ttggtgggttgcaaagatgcttgggggtcttctccataggggtcataagataaaagggacagtggtgttgatgcgcaagaatgtgttggacgtgaatagcgtaaccagcgttgggggaattattggtcaaggtctcgacttagttggctcaacactcgatactcttactgccttcttgggccgatccgtgtctctccagcttattagtgctaccaaagctgatgccaacggaaaagggaaacttggaaaggctacctttttggaaggtatcattacttcattgccaactttgggagcaggccaatctgcattcaaaattaattttgaatgggatgatgggagtggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatgaaggtgttgaaagttctatttggctgctagcaaaggcttatgtagttgtgaatgactcgtgctatcatcaacttgtcagccattggttaaacactcatgcagttgttgagccattcatcatagcaacaaacaggcatctcagtgttgttcaccctatttataaactccttcaccctcactatcgtgacaccatgaacataaatggccttgctcggttatcactggtcaacgacggtggcgttatagaacaaacatttttgtggggaaggtattctgtggaaatgtctgctgtagtttacaaggattgggtttttacagatcaagcattgcctgctgatcttataaaaagaggaatggcaattgaggatccatcgtgccctcatggcattcgccttgtgatagaggactacccttatgctgttgatggacttgagatatgggatgctatcaagacatgggtccatgaatacgttttcttgtactacaaatcagatgacacacttagagaagatcctgaactccaagcctgctggaaagaactcgtagaggtgggtcatggagacaagaaaaatgagccatggtggcctaagatgcaaactcgtgaagagctagttgaagcttgcgctatcatcatatggactgcttcagcacttcatgcagctgttaattttggacagtatccctatggaggtttaatcttaaaccgtccaactcttagtaggcgattcatgcctgagaaaggttctgctgagtatgaggagctgaggaagaatccccagaaggcttacttgaagactattacaccaaagtttcagacccttattgacctttctgttatagaaatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggaattcccaacagtatctctatctgaagaggtttgtggttccttaagttttgtggctttgaataaaagatagaagagaaggaatctgaatttcttcatccaacctgcacatggtaatctagtgtgtttgagtgctttcatgtttggctttaataaaataaaagcagttgttttgtaaaatgttcagaatcaataaaagcatttactgctttaaaaaaaaaaaaaaaaaaa

SEQ ID NO: 49 - DNA, 12678 bp, Artificial
  plasmid PHP22905
ggccgccggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgttcccacccagtgagagaaaaggaatagcaatatcaacactgtgttgggctaccatgttttctctgcttatctatctctcattcataactagtccacttctagtgctcaagctctatggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagggcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtaccggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgc

SEQ ID NO: 50 - DNA, 12678 bp, Artificial
  plasmid PHP22972
ggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtaccggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgccctctgaaagttaatccttccttactagaaggcaaaagcagagtataaggcatttgaactggtccacaacggtttctcagtttctcatcgttgtttctctctgagagtttattctcaatttgtgccagtttatttccaaaccttttaaaagcctctaatgctcttgtatcagatgtccaatttggattgtccctctccccaaggtacacctcatcagatgcatgccttgacaagatctctatcactgaaaggtcaactagagtttgaaactttgatgtaatagttctcaaataagccttttgatgacttttcaccatttcttcatattctggggtgcctttctcaggaagcaaccttctagaagaagttgggcgattcagaatgaaacctccatatggatattgaccaaagttaacggctgcatggagggctgaagcagtccatatgataatggtgcaaatttcaacaagctcttcaattgtttgcaacttaggccaccatggcttgtctttcagatcaccatgacctttctctacagcttctttccaccactgttggagttcagaatcaggtttgacatcatcatctcttgcatagtacaaggacacatattcttggacccatgtcttaattgcagcccatatctctagcccatcaacagcataagggtagtcctctatcagaagtcgaagtccatgtggggcagatggatccttaattgccactcctctcttgataagatctgcaggcagaaaaacttgactaaaagcaccagattgatctccctgaggatgtggcaaactcaactctatggcaagtggccttaaagttccgtcatttttcaggaaaaggatggttctggtagcataagcctttgtggaggttgcatttattcgcctcaaatatggcatgattgggtcatgatgatctagtaggaacaatctcttgttttgaattgcctcatctacagtgagcccttctaagttgggctctaggtgttctttggttatttgactagtatgatcaccatagacttggctatctagcttgcttcgtggagggaaatccttaagacaacgaatcaagtttggatttacaccagcaagcatttctcttgcaaattcttcatcagtcatccatgcagacttactcacttgaattactttaggaggaggaaacttaagggcctgttcaccatcagttcggaagatttccttaagcacgggtagtggactaatcttgctgattatatctgttggcagcttaattccgcctgaatagagtccatgaacttcatcaaagctatcaaactcacggggtgtgaaattcaaatcaaaagcagattgcaataatggaagaacattttgagatacggattttagtccataagtaagaaagtcagatgacttcaagtgtccaaaagcctcatctcttggaagataaacatcattgctcctactctcactattaggatctttccttgttggttttctaccagttctccccctacgaggataaggaaaggtgtcatttcctccaagaacaggacgggcatgattttcacctttatccggatcacctaaatcattgtagacatcataatcatagatcctttcccactctttgcgttctccagttccatctcctcttaaattatgcaactcttcttctctatatttgactagtggagctggtgtctcacttggaagatatgtctggttggcaaagaaaatgcggtcacttttgaagagtttggcattgtaaatccacgaattgcaaacaaagtggatgcttccatggtttggaatgtcttcaagagtcaaactcacaaggaaaaactcagtttgcataaaattcttgatataaaatgctccaggaattccatagagcttgagcactagaagtggactagttatgaatgagagatagataagcagagaaaacatggtagcccaacacagtgttgatattgctattccttttctctcactgggtgggaacagattgctgtagggattgaagtgagagccttcctttccggggcttcttgaaaacaaataaattggatacacaaacaatggaaatggcacagtgaatctaatgagtcttgtcatgctgtctagattcttgtaaatcttctctgttaatggaacccatgactcatccttctcaatgtgtccatggttttgatggtgagttctgtggctaattctccatccatggtatggcacaagaattgaggaatgcaagatgtgtcccaccaggctattcagcaaagggctatctgaaaagcttccatggccacaatcatgtccaagaacaaagagagcccagaacattgtgccttgaatggggcaatagattagccagagaagccagttgtcgaagtgaattgctgcagccaccaatgcagcaattacaagcacatccctgagaacataactgagggatctccatggattcttgacccagcaatgttttggtattgaagctctgatttctgcaatcttaaacggtggaggagcccttctcggatgttccttcatctggctccacatagaggcactctctcgcttctctccacagtgccttgtaaaatggtgtgtcatcaaattggtagtactcacccaatattggcttgattgcattggttgcctccattgcatggtaatgtggcattgtagagaagagatggtgagccacatgagtatcagttatgtgatgaaacaccttgttcagaatcccataatctctgtccatagttgccaaagctcccttcagccagtcccattctgatgaatcgtaatgaggcaaggcaaagtgtgtgtgctgcaaatatgtgatagtcacaagaaaaccgttcacaatgagcaaaggcaccccataaacacatagcagccaaaccaaccctttcagggttgcaacacggtagagagagtaagtcctgcaagtactcctgcatctgatatatatatttgaagtcgttcacgatcagagtaaatgggaccatatgggtcatagtggcaagcaaatctatcataaggccttccagaaacatttaaagccaagtacaagggccaaccaagtgtgagggtgacagcaagagtgaggactctgcctggaggattgttaaggtatttagagtaccacttgatacaggacttctgctttggcacaaatacttcatcccgctcaagagaaccagtgttggagtggtgacggcgatggctgtatttccatgaaaagtacgggactaggagagcggagtggaggataaggccaacaatatcatcaagcagctggtagtcactgaatgcatggtgaccacactcatgggcaatgacccaaacaccagtaaggatgcaaccctggacagcccaatagattgccatgccacggaaagagagaggaccggc

SEQ ID NO: 51 - DNA, 10164 bp, Artificial
  recombinant DNA fragment PHP22905A
cgcgccggtaccgggccccccctcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgccggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgttcccacccagtgagagaaaaggaatagcaatatcaacactgtgttgggctaccatgttttctctgcttatctatctctcattcataactagtccacttctagtgctcaagctctatggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagggcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtaccgg

SEQ ID NO: 52 - DNA, 10164 bp, Artificial
  recombinant DNA fragment PHP22972A
cgcgccggtaccgggccccccctcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgccctctgaaagttaatccttccttactagaaggcaaaagcagagtataaggcatttgaactggtccacaacggtttctcagtttctcatcgttgtttctctctgagagtttattctcaatttgtgccagtttatttccaaaccttttaaaagcctctaatgctcttgtatcagatgtccaatttggattgtccctctccccaaggtacacctcatcagatgcatgccttgacaagatctctatcactgaaaggtcaactagagtttgaaactttgatgtaatagttctcaaataagccttttgatgacttttcaccatttcttcatattctggggtgcctttctcaggaagcaaccttctagaagaagttgggcgattcagaatgaaacctccatatggatattgaccaaagttaacggctgcatggagggctgaagcagtccatatgataatggtgcaaatttcaacaagctcttcaattgtttgcaacttaggccaccatggcttgtctttcagatcaccatgacctttctctacagcttctttccaccactgttggagttcagaatcaggtttgacatcatcatctcttgcatagtacaaggacacatattcttggacccatgtcttaattgcagcccatatctctagcccatcaacagcataagggtagtcctctatcagaagtcgaagtccatgtggggcagatggatccttaattgccactcctctcttgataagatctgcaggcagaaaaacttgactaaaagcaccagattgatctccctgaggatgtggcaaactcaactctatggcaagtggccttaaagttccgtcatttttcaggaaaaggatggttctggtagcataagcctttgtggaggttgcatttattcgcctcaaatatggcatgattgggtcatgatgatctagtaggaacaatctcttgttttgaattgcctcatctacagtgagcccttctaagttgggctctaggtgttctttggttatttgactagtatgatcaccatagacttggctatctagcttgcttcgtggagggaaatccttaagacaacgaatcaagtttggatttacaccagcaagcatttctcttgcaaattcttcatcagtcatccatgcagacttactcacttgaattactttaggaggaggaaacttaagggcctgttcaccatcagttcggaagatttccttaagcacgggtagtggactaatcttgctgattatatctgttggcagcttaattccgcctgaatagagtccatgaacttcatcaaagctatcaaactcacggggtgtgaaattcaaatcaaaagcagattgcaataatggaagaacattttgagatacggattttagtccataagtaagaaagtcagatgacttcaagtgtccaaaagcctcatctcttggaagataaacatcattgctcctactctcactattaggatctttccttgttggttttctaccagttctccccctacgaggataaggaaaggtgtcatttcctccaagaacaggacgggcatgattttcacctttatccggatcacctaaatcattgtagacatcataatcatagatcctttcccactctttgcgttctccagttccatctcctcttaaattatgcaactcttcttctctatatttgactagtggagctggtgtctcacttggaagatatgtctggttggcaaagaaaatgcggtcacttttgaagagtttggcattgtaaatccacgaattgcaaacaaagtggatgcttccatggtttggaatgtcttcaagagtcaaactcacaaggaaaaactcagtttgcataaaattcttgatataaaatgctccaggaattccatagagcttgagcactagaagtggactagttatgaatgagagatagataagcagagaaaacatggtagcccaacacagtgttgatattgctattccttttctctcactgggtgggaacagattgctgtagggattgaagtgagagccttcctttccggggcttcttgaaaacaaataaattggatacacaaacaatggaaatggcacagtgaatctaatgagtcttgtcatgctgtctagattcttgtaaatcttctctgttaatggaacccatgactcatccttctcaatgtgtccatggttttgatggtgagttctgtggctaattctccatccatggtatggcacaagaattgaggaatgcaagatgtgtcccaccaggctattcagcaaagggctatctgaaaagcttccatggccacaatcatgtccaagaacaaagagagcccagaacattgtgccttgaatggggcaatagattagccagagaagccagttgtcgaagtgaattgctgcagccaccaatgcagcaattacaagcacatccctgagaacataactgagggatctccatggattcttgacccagcaatgttttggtattgaagctctgatttctgcaatcttaaacggtggaggagcccttctcggatgttccttcatctggctccacatagaggcactctctcgcttctctccacagtgccttgtaaaatggtgtgtcatcaaattggtagtactcacccaatattggcttgattgcattggttgcctccattgcatggtaatgtggcattgtagagaagagatggtgagccacatgagtatcagttatgtgatgaaacaccttgttcagaatcccataatctctgtccatagttgccaaagctcccttcagccagtcccattctgatgaatcgtaatgaggcaaggcaaagtgtgtgtgctgcaaatatgtgatagtcacaagaaaaccgttcacaatgagcaaaggcaccccataaacacatagcagccaaaccaaccctttcagggttgcaacacggtagagagagtaagtcctgcaagtactcctgcatctgatatatatatttgaagtcgttcacgatcagagtaaatgggaccatatgggtcatagtggcaagcaaatctatcataaggccttccagaaacatttaaagccaagtacaagggccaaccaagtgtgagggtgacagcaagagtgaggactctgcctggaggattgttaaggtatttagagtaccacttgatacaggacttctgctttggcacaaatacttcatcccgctcaagagaaccagtgttggagtggtgacggcgatggctgtatttccatgaaaagtacgggactaggagagcggagtggaggataaggccaacaatatcatcaagcagctggtagtcactgaatgcatggtgaccacactcatgggcaatgacccaaacaccagtaaggatgcaaccctggacagcccaatagattgccatgccacggaaagagagaggaccggcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtaccgg

SEQ ID NO: 53 - protein, 500 aa, Euphorbia lagascae
MEQKNLSFPSILISFLLVLILVVVMRLWKKQNPPPGPWKFPIIGNLPHLLLTSDLGHERFRALAQIYGPVMSLQIGQVSAVVISSAEAAKEVMKTQADAFAQRPIVLDAQIVFYNRKDVLFASYGDHWRQMKKIWILEFLSAKKVQSSRLIREEEMEDAITFLRSKAGSPVNITKIIYGIIISIMIRTSVGNCKQKERLLSVADAVNEAATSFGTADAFPTWKLLHYIIGAESKPRRLHQEIDDILEEILNEHKANKPFEADNLMDVLLNLQKNGNVPVPVTNESIKASVLQMFTAGSETTSKATEWVMAELMKNPTELRKAQEEVRQVFGEMGKVDESRFHDLKFFKLVVKETLRLHPPVVLIPRECRETTRIDGYEIHPNTRIVVNAWAIGRDPNTWSEPGKFNPERFKDCAIDYKGTTFELVPFGAGKRICPGITSAITNLEYVIINLLYHFNWELADGITPQTLDMTEAIGGALRKKIDLKLIPIPYQVSLGSNIS

SEQ ID NO: 54 - DNA, 1784 bp, Glycine max
gcacgagtttagttcttttccatggctctattattcttctactttttggttcttatttcctttgccttcacaaccattatagtacaaaaaataagaaagaaacccaagaaaactgatgacacaacttgtaaaataccccatggtcctagaaagctacctattataggaaatatatacaatctgctatgctctcaaccccatagaaaattaagagacctggccataaaatatggacccgtgatgcatcttcaacttggacaggtttcaactattgtcatttcatcccctgagtgtgctagggaagtgatgaaaacccatgacattaactttgccacaaggcctaaagttctagctattgaaataatgtcttacaattccacaagtatagcttttgctggctatggaaattattggaggcagctaagaaaaatatgcacattggagcttttaagcctaaaacgcgtcaactcattccagccaattagagaagatgagctcttcaatcttgtcaaatggattgattcaaagaaaggatcccccatcaacctcactgaagcagtacttacatcaatttacacaattgcttcaagggctgcctttggcaagaattgcaaagaccaagaaaaatttatatcagtggttaaaaaaacatcaaaacttgctgcaggttttggcattgaagatttgtttccttctgttacttggcttcaacatgtcactggcttgagggctaagcttgagaggttgcatcaacagactgatcagataatggaaaacatcatcaatgagcataaagaggcaaattcgaaagccaaggatgaccaaagtgaagcagaagaagatcttgtggatgttctcatacaatatgaggatgggagcaagaaggatttttccttaactagaaacaagatcaaggccataattctggacatttttgctgctggaggtgagacaacagcaacaaccatagattgggcaatggctgaaatggtaaagaatccaacagtaatgaagaaagcacaatctgaggtgagagaggtatgcaatatgaaagcaagggttgatgaaaattgcatcaatgaacttcaatatttgaaactaattgtgaaagagaccttgaggttgcaccctccagctcctcttttgctgccaagagaatgtggtcaaacatgtgagatacatggttatcacataccagccaaaaccaaggtcatagtcaatgcctgggcaattggaagagatccaaactattggactgaatcagagaggttttatcctgagaggttcattgatagcactattgactacaaagggagtaattttgagttcattccttttggtgctggaagaaggatatgcgcaggaagcacatttgctttgagagctgcggaactagcccttgcaatgttgttgtatcactttgattggaagcttccaagtggaatgagaagtggagaactggacatgagtgaggattttggagtcacaactataagaaaagataatctattcttggttccttttccatatcatcctttgcctgtctcatgagctcagaatgagaaactgaaacatttggtgatggctgtcacatttacttcagaataaagatcttctgttaattgaaattagactttgtgctgtgattggttgtaacagaagatggaaattgtgactactgcatattaaaatagcttttatatatcctcaatgttaattaactttgataaaaaaaaaaaaaaaaaaaaaaaaaaaaaaaaaaaaaaaaaaaaaaaaaaaaaaaa

SEQ ID NO: 55 - protein, 509 aa, Glycine max
MALLFFYFLVLISFAFTTIIVQKIRKKPKKTDDTTCKIPHGPRKLPIIGNIYNLLCSQPHRKLRDLAIKYGPVMHLQLGQVSTIVISSPECAREVMKTHDINFATRPKVLAIEIMSYNSTSIAFAGYGNYWRQLRKICTLELLSLKRVNSFQPIREDELFNLVKWIDSKKGSPINLTEAVLTSIYTIASRAAFGKNCKDQEKFISVVKKTSKLAAGFGIEDLFPSVTWLQHVTGLRAKLERLHQQTDQIMENIINEHKEANSKAKDDQSEAEEDLVDVLIQYEDGSKKDFSLTRNKIKAIILDIFAAGGETTATTIDWAMAEMVKNPTVMKKAQSEVREVCNMKARVDENCINELQYLKLIVKETLRLHPPAPLLLPRECGQTCEIHGYHIPAKTKVIVNAWAIGRDPNYWTESERFYPERFIDSTIDYKGSNFEFIPFGAGRRICAGSTFALRAAELALAMLLYHFDWKLPSGMRSGELDMSEDFGVTTIRKDNLFLVPFPYHPLPVS

SEQ ID NO: 56 - DNA, 35 bp, Artificial
  primer
gcggccgcatggctctattattcttctactttttg

SEQ ID NO: 57 - DNA, 46 bp, Artificial
  primer
cttgatataaaatgctccaggaattcaacctcaaggtctctttcac

SEQ ID NO: 58 - DNA, 46 bp, Artificial
  primer
gtgaaagagaccttgaggttgaattcctggagcattttatatcaag

SEQ ID NO: 59 - DNA, 29 bp, Artificial
  primer
gcggccgccctctgaaagttaatccttcc

SEQ ID NO: 60 - DNA, 3001 bp, Artificial
  sequence comprising a portion of the P450-EPOX gene and portions of the LOX2 and LOX3 genes
gcggccgcatggctctattattcttctactttttggttcttatttcctttgccttcacaaccattatagtacaaaaaataagaaagaaacccaagaaaactgatgacacaacttgtaaaataccccatggtcctagaaagctacctattataggaaatatatacaatctgctatgctctcaaccccatagaaaattaagagacctggccataaaatatggacccgtgatgcatcttcaacttggacaggtttcaactattgtcatttcatcccctgagtgtgctagggaagtgatgaaaacccatgacattaactttgccacaaggcctaaagttctagctattgaaataatgtcttacaattccacaagtatagcttttgctggctatggaaattattggaggcagctaagaaaaatatgcacattggagcttttaagcctaaaacgcgtcaactcattccagccaattagagaagatgagctcttcaatcttgtcaaatggattgattcaaagaaaggatcccccatcaacctcactgaagcagtacttacatcaatttacacaattgcttcaagggctgcctttggcaagaattgcaaagaccaagaaaaatttatatcagtggttaaaaaaacatcaaaacttgctgcaggttttggcattgaagatttgtttccttctgttacttggcttcaacatgtcactggcttgagggctaagcttgagaggttgcatcaacagactgatcagataatggaaaacatcatcaatgagcataaagaggcaaattcgaaagccaaggatgaccaaagtgaagcagaagaagatcttgtggatgttctcatacaatatgaggatgggagcaagaaggatttttccttaactagaaacaagatcaaggccataattctggacatttttgctgctggaggtgagacaacagcaacaaccatagattgggcaatggctgaaatggtaaagaatccaacagtaatgaagaaagcacaatctgaggtgagagaggtatgcaatatgaaagcaagggttgatgaaaattgcatcaatgaacttcaatatttgaaactaattgtgaaagagaccttgaggttgaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagggcggccgc

SEQ ID NO: 61 - DNA, 6914 bp, Artificial
  sequence comprising SEQ ID NO60 cloned into pPCR2.1
aagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaattcagggcgcaagggctgctaaaggaaccggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcgccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgatccatggcgatgcctgcttgccgaatatcatggtggaaaatggccgcttttctggattcaacgactgtggccggctgggtgtggcggaccgctatcaggacatagcgttggatacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgcccttggtacgcggccgcatggctctattattcttctactttttggttcttatttcctttgccttcacaaccattatagtacaaaaaataagaaagaaacccaagaaaactgatgacacaacttgtaaaataccccatggtcctagaaagctacctattataggaaatatatacaatctgctatgctctcaaccccatagaaaattaagagacctggccataaaatatggacccgtgatgcatcttcaacttggacaggtttcaactattgtcatttcatcccctgagtgtgctagggaagtgatgaaaacccatgacattaactttgccacaaggcctaaagttctagctattgaaataatgtcttacaattccacaagtatagcttttgctggctatggaaattattggaggcagctaagaaaaatatgcacattggagcttttaagcctaaaacgcgtcaactcattccagccaattagagaagatgagctcttcaatcttgtcaaatggattgattcaaagaaaggatcccccatcaacctcactgaagcagtacttacatcaatttacacaattgcttcaagggctgcctttggcaagaattgcaaagaccaagaaaaatttatatcagtggttaaaaaaacatcaaaacttgctgcaggttttggcattgaagatttgtttccttctgttacttggcttcaacatgtcactggcttgagggctaagcttgagaggttgcatcaacagactgatcagataatggaaaacatcatcaatgagcataaagaggcaaattcgaaagccaaggatgaccaaagtgaagcagaagaagatcttgtggatgttctcatacaatatgaggatgggagcaagaaggatttttccttaactagaaacaagatcaaggccataattctggacatttttgctgctggaggtgagacaacagcaacaaccatagattgggcaatggctgaaatggtaaagaatccaacagtaatgaagaaagcacaatctgaggtgagagaggtatgcaatatgaaagcaagggttgatgaaaattgcatcaatgaacttcaatatttgaaactaattgtgaaagagaccttgaggttgaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagggcggccgc

SEQ ID NO: 62 - DNA, 12249 bp, Artificial
  plasmid PHP23466
ggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtaccggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgccctctgaaagttaatccttccttactagaaggcaaaagcagagtataaggcatttgaactggtccacaacggtttctcagtttctcatcgttgtttctctctgagagtttattctcaatttgtgccagtttatttccaaaccttttaaaagcctctaatgctcttgtatcagatgtccaatttggattgtccctctccccaaggtacacctcatcagatgcatgccttgacaagatctctatcactgaaaggtcaactagagtttgaaactttgatgtaatagttctcaaataagccttttgatgacttttcaccatttcttcatattctggggtgcctttctcaggaagcaaccttctagaagaagttgggcgattcagaatgaaacctccatatggatattgaccaaagttaacggctgcatggagggctgaagcagtccatatgataatggtgcaaatttcaacaagctcttcaattgtttgcaacttaggccaccatggcttgtctttcagatcaccatgacctttctctacagcttctttccaccactgttggagttcagaatcaggtttgacatcatcatctcttgcatagtacaaggacacatattcttggacccatgtcttaattgcagcccatatctctagcccatcaacagcataagggtagtcctctatcagaagtcgaagtccatgtggggcagatggatccttaattgccactcctctcttgataagatctgcaggcagaaaaacttgactaaaagcaccagattgatctccctgaggatgtggcaaactcaactctatggcaagtggccttaaagttccgtcatttttcaggaaaaggatggttctggtagcataagcctttgtggaggttgcatttattcgcctcaaatatggcatgattgggtcatgatgatctagtaggaacaatctcttgttttgaattgcctcatctacagtgagcccttctaagttgggctctaggtgttctttggttatttgactagtatgatcaccatagacttggctatctagcttgcttcgtggagggaaatccttaagacaacgaatcaagtttggatttacaccagcaagcatttctcttgcaaattcttcatcagtcatccatgcagacttactcacttgaattactttaggaggaggaaacttaagggcctgttcaccatcagttcggaagatttccttaagcacgggtagtggactaatcttgctgattatatctgttggcagcttaattccgcctgaatagagtccatgaacttcatcaaagctatcaaactcacggggtgtgaaattcaaatcaaaagcagattgcaataatggaagaacattttgagatacggattttagtccataagtaagaaagtcagatgacttcaagtgtccaaaagcctcatctcttggaagataaacatcattgctcctactctcactattaggatctttccttgttggttttctaccagttctccccctacgaggataaggaaaggtgtcatttcctccaagaacaggacgggcatgattttcacctttatccggatcacctaaatcattgtagacatcataatcatagatcctttcccactctttgcgttctccagttccatctcctcttaaattatgcaactcttcttctctatatttgactagtggagctggtgtctcacttggaagatatgtctggttggcaaagaaaatgcggtcacttttgaagagtttggcattgtaaatccacgaattgcaaacaaagtggatgcttccatggtttggaatgtcttcaagagtcaaactcacaaggaaaaactcagtttgcataaaattcttgatataaaatgctccaggaattcaacctcaaggtctctttcacaattagtttcaaatattgaagttcattgatgcaattttcatcaacccttgctttcatattgcatacctctctcacctcagattgtgctttcttcattactgttggattctttaccatttcagccattgcccaatctatggttgttgctgttgtctcacctccagcagcaaaaatgtccagaattatggccttgatcttgtttctagttaaggaaaaatccttcttgctcccatcctcatattgtatgagaacatccacaagatcttcttctgcttcactttggtcatccttggctttcgaatttgcctctttatgctcattgatgatgttttccattatctgatcagtctgttgatgcaacctctcaagcttagccctcaagccagtgacatgttgaagccaagtaacagaaggaaacaaatcttcaatgccaaaacctgcagcaagttttgatgtttttttaaccactgatataaatttttcttggtctttgcaattcttgccaaaggcagcccttgaagcaattgtgtaaattgatgtaagtactgcttcagtgaggttgatgggggatcctttctttgaatcaatccatttgacaagattgaagagctcatcttctctaattggctggaatgagttgacgcgttttaggcttaaaagctccaatgtgcatatttttcttagctgcctccaataatttccatagccagcaaaagctatacttgtggaattgtaagacattatttcaatagctagaactttaggccttgtggcaaagttaatgtcatgggttttcatcacttccctagcacactcaggggatgaaatgacaatagttgaaacctgtccaagttgaagatgcatcacgggtccatattttatggccaggtctcttaattttctatggggttgagagcatagcagattgtatatatttcctataataggtagctttctaggaccatggggtattttacaagttgtgtcatcagttttcttgggtttctttcttattttttgtactataatggttgtgaaggcaaaggaaataagaaccaaaaagtagaagaataatagagccatgc

SEQ ID NO: 63 - DNA, 9735 bp, Artificial
  recombinant DNA fragment PHP23466A
cgcgccggtaccgggccccccctcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgccctctgaaagttaatccttccttactagaaggcaaaagcagagtataaggcatttgaactggtccacaacggtttctcagtttctcatcgttgtttctctctgagagtttattctcaatttgtgccagtttatttccaaaccttttaaaagcctctaatgctcttgtatcagatgtccaatttggattgtccctctccccaaggtacacctcatcagatgcatgccttgacaagatctctatcactgaaaggtcaactagagtttgaaactttgatgtaatagttctcaaataagccttttgatgacttttcaccatttcttcatattctggggtgcctttctcaggaagcaaccttctagaagaagttgggcgattcagaatgaaacctccatatggatattgaccaaagttaacggctgcatggagggctgaagcagtccatatgataatggtgcaaatttcaacaagctcttcaattgtttgcaacttaggccaccatggcttgtctttcagatcaccatgacctttctctacagcttctttccaccactgttggagttcagaatcaggtttgacatcatcatctcttgcatagtacaaggacacatattcttggacccatgtcttaattgcagcccatatctctagcccatcaacagcataagggtagtcctctatcagaagtcgaagtccatgtggggcagatggatccttaattgccactcctctcttgataagatctgcaggcagaaaaacttgactaaaagcaccagattgatctccctgaggatgtggcaaactcaactctatggcaagtggccttaaagttccgtcatttttcaggaaaaggatggttctggtagcataagcctttgtggaggttgcatttattcgcctcaaatatggcatgattgggtcatgatgatctagtaggaacaatctcttgttttgaattgcctcatctacagtgagcccttctaagttgggctctaggtgttctttggttatttgactagtatgatcaccatagacttggctatctagcttgcttcgtggagggaaatccttaagacaacgaatcaagtttggatttacaccagcaagcatttctcttgcaaattcttcatcagtcatccatgcagacttactcacttgaattactttaggaggaggaaacttaagggcctgttcaccatcagttcggaagatttccttaagcacgggtagtggactaatcttgctgattatatctgttggcagcttaattccgcctgaatagagtccatgaacttcatcaaagctatcaaactcacggggtgtgaaattcaaatcaaaagcagattgcaataatggaagaacattttgagatacggattttagtccataagtaagaaagtcagatgacttcaagtgtccaaaagcctcatctcttggaagataaacatcattgctcctactctcactattaggatctttccttgttggttttctaccagttctccccctacgaggataaggaaaggtgtcatttcctccaagaacaggacgggcatgattttcacctttatccggatcacctaaatcattgtagacatcataatcatagatcctttcccactctttgcgttctccagttccatctcctcttaaattatgcaactcttcttctctatatttgactagtggagctggtgtctcacttggaagatatgtctggttggcaaagaaaatgcggtcacttttgaagagtttggcattgtaaatccacgaattgcaaacaaagtggatgcttccatggtttggaatgtcttcaagagtcaaactcacaaggaaaaactcagtttgcataaaattcttgatataaaatgctccaggaattcaacctcaaggtctctttcacaattagtttcaaatattgaagttcattgatgcaattttcatcaacccttgctttcatattgcatacctctctcacctcagattgtgctttcttcattactgttggattctttaccatttcagccattgcccaatctatggttgttgctgttgtctcacctccagcagcaaaaatgtccagaattatggccttgatcttgtttctagttaaggaaaaatccttcttgctcccatcctcatattgtatgagaacatccacaagatcttcttctgcttcactttggtcatccttggctttcgaatttgcctctttatgctcattgatgatgttttccattatctgatcagtctgttgatgcaacctctcaagcttagccctcaagccagtgacatgttgaagccaagtaacagaaggaaacaaatcttcaatgccaaaacctgcagcaagttttgatgtttttttaaccactgatataaatttttcttggtctttgcaattcttgccaaaggcagcccttgaagcaattgtgtaaattgatgtaagtactgcttcagtgaggttgatgggggatcctttctttgaatcaatccatttgacaagattgaagagctcatcttctctaattggctggaatgagttgacgcgttttaggcttaaaagctccaatgtgcatatttttcttagctgcctccaataatttccatagccagcaaaagctatacttgtggaattgtaagacattatttcaatagctagaactttaggccttgtggcaaagttaatgtcatgggttttcatcacttccctagcacactcaggggatgaaatgacaatagttgaaacctgtccaagttgaagatgcatcacgggtccatattttatggccaggtctcttaattttctatggggttgagagcatagcagattgtatatatttcctataataggtagctttctaggaccatggggtattttacaagttgtgtcatcagttttcttgggtttctttcttattttttgtactataatggttgtgaaggcaaaggaaataagaaccaaaaagtagaagaataatagagccatgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtaccgg

SEQ ID NO: 64 - DNA, 5031 bp, Artificial
  sequence comprising a 1100 bp fragment of clone sfl1.pk0045.g7 inserted into plasmid pCR2.1
aagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaattcagggcgcaagggctgctaaaggaaccggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcgccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgatccatggcgatgcctgcttgccgaatatcatggtggaaaatggccgcttttctggattcaacgactgtggccggctgggtgtggcggaccgctatcaggacatagcgttggatacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgcccttggtacgcggccgcatggctctattattcttctactttttggttcttatttcctttgccttcacaaccattatagtacaaaaaataagaaagaaacccaagaaaactgatgacacaacttgtaaaataccccatggtcctagaaagctacctattataggaaatatatacaatctgctatgctctcaaccccatagaaaattaagagacctggccataaaatatggacccgtgatgcatcttcaacttggacaggtttcaactattgtcatttcatcccctgagtgtgctagggaagtgatgaaaacccatgacattaactttgccacaaggcctaaagttctagctattgaaataatgtcttacaattccacaagtatagcttttgctggctatggaaattattggaggcagctaagaaaaatatgcacattggagcttttaagcctaaaacgcgtcaactcattccagccaattagagaagatgagctcttcaatcttgtcaaatggattgattcaaagaaaggatcccccatcaacctcactgaagcagtacttacatcaatttacacaattgcttcaagggctgcctttggcaagaattgcaaagaccaagaaaaatttatatcagtggttaaaaaaacatcaaaacttgctgcaggttttggcattgaagatttgtttccttctgttacttggcttcaacatgtcactggcttgagggctaagcttgagaggttgcatcaacagactgatcagataatggaaaacatcatcaatgagcataaagaggcaaattcgaaagccaaggatgaccaaagtgaagcagaagaagatcttgtggatgttctcatacaatatgaggatgggagcaagaaggatttttccttaactagaaacaagatcaaggccataattctggacatttttgctgctggaggtgagacaacagcaacaaccatagattgggcaatggctgaaatggtaaagaatccaacagtaatgaagaaagcacaatctgaggtgagagaggtatgcaatatgaaagcaagggttgatgaaaattgcatcaatgaacttcaatatttgaaactaattgtgaaagagaccttgaggttgctcgag

SEQ ID NO: 65 - DNA, 33 bp, Artificial
  primer
ctcgagcggtcctctctctttccgtggcatggc

SEQ ID NO: 66 - DNA, 7341 bp, Artificial
  sequence comprising a portion of the FAD2-2 gene, a portion of the FAD2-1 gene, a portion of the FAD3 gene, and portions of the LOX2 and LOX3 genes inserted into plasmid pCR2.1
aagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaattcagggcgcaagggctgctaaaggaaccggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcgccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgatccatggcgatgcctgcttgccgaatatcatggtggaaaatggccgcttttctggattcaacgactgtggccggctgggtgtggcggaccgctatcaggacatagcgttggatacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgcccttggtacctcgagcggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgttcccacccagtgagagaaaaggaatagcaatatcaacactgtgttgggctaccatgttttctctgcttatctatctctcattcataactagtccacttctagtgctcaagctctatggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagggcggccgc

SEQ ID NO: 67 - DNA, 13788 bp, Artificial
  plasmid PHP23465
ggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtaccggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcatggctctattattcttctactttttggttcttatttcctttgccttcacaaccattatagtacaaaaaataagaaagaaacccaagaaaactgatgacacaacttgtaaaataccccatggtcctagaaagctacctattataggaaatatatacaatctgctatgctctcaaccccatagaaaattaagagacctggccataaaatatggacccgtgatgcatcttcaacttggacaggtttcaactattgtcatttcatcccctgagtgtgctagggaagtgatgaaaacccatgacattaactttgccacaaggcctaaagttctagctattgaaataatgtcttacaattccacaagtatagcttttgctggctatggaaattattggaggcagctaagaaaaatatgcacattggagcttttaagcctaaaacgcgtcaactcattccagccaattagagaagatgagctcttcaatcttgtcaaatggattgattcaaagaaaggatcccccatcaacctcactgaagcagtacttacatcaatttacacaattgcttcaagggctgcctttggcaagaattgcaaagaccaagaaaaatttatatcagtggttaaaaaaacatcaaaacttgctgcaggttttggcattgaagatttgtttccttctgttacttggcttcaacatgtcactggcttgagggctaagcttgagaggttgcatcaacagactgatcagataatggaaaacatcatcaatgagcataaagaggcaaattcgaaagccaaggatgaccaaagtgaagcagaagaagatcttgtggatgttctcatacaatatgaggatgggagcaagaaggatttttccttaactagaaacaagatcaaggccataattctggacatttttgctgctggaggtgagacaacagcaacaaccatagattgggcaatggctgaaatggtaaagaatccaacagtaatgaagaaagcacaatctgaggtgagagaggtatgcaatatgaaagcaagggttgatgaaaattgcatcaatgaacttcaatatttgaaactaattgtgaaagagaccttgaggttgctcgagcggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgttcccacccagtgagagaaaaggaatagcaatatcaacactgtgttgggctaccatgttttctctgcttatctatctctcattcataactagtccacttctagtgctcaagctctatggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagggc

SEQ ID NO: 68 - DNA, 11274 bp, Artificial
  recombinant DNA fragment PHP23465A
cgcgccggtaccgggccccccctcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcatggctctattattcttctactttttggttcttatttcctttgccttcacaaccattatagtacaaaaaataagaaagaaacccaagaaaactgatgacacaacttgtaaaataccccatggtcctagaaagctacctattataggaaatatatacaatctgctatgctctcaaccccatagaaaattaagagacctggccataaaatatggacccgtgatgcatcttcaacttggacaggtttcaactattgtcatttcatcccctgagtgtgctagggaagtgatgaaaacccatgacattaactttgccacaaggcctaaagttctagctattgaaataatgtcttacaattccacaagtatagcttttgctggctatggaaattattggaggcagctaagaaaaatatgcacattggagcttttaagcctaaaacgcgtcaactcattccagccaattagagaagatgagctcttcaatcttgtcaaatggattgattcaaagaaaggatcccccatcaacctcactgaagcagtacttacatcaatttacacaattgcttcaagggctgcctttggcaagaattgcaaagaccaagaaaaatttatatcagtggttaaaaaaacatcaaaacttgctgcaggttttggcattgaagatttgtttccttctgttacttggcttcaacatgtcactggcttgagggctaagcttgagaggttgcatcaacagactgatcagataatggaaaacatcatcaatgagcataaagaggcaaattcgaaagccaaggatgaccaaagtgaagcagaagaagatcttgtggatgttctcatacaatatgaggatgggagcaagaaggatttttccttaactagaaacaagatcaaggccataattctggacatttttgctgctggaggtgagacaacagcaacaaccatagattgggcaatggctgaaatggtaaagaatccaacagtaatgaagaaagcacaatctgaggtgagagaggtatgcaatatgaaagcaagggttgatgaaaattgcatcaatgaacttcaatatttgaaactaattgtgaaagagaccttgaggttgctcgagcggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgttcccacccagtgagagaaaaggaatagcaatatcaacactgtgttgggctaccatgttttctctgcttatctatctctcattcataactagtccacttctagtgctcaagctctatggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagggcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtaccgg

SEQ ID NO: 69 - DNA, 8031 bp, Artificial
  sequence resulting from digestion of pDS3 (orientation 2) with Not 1
ggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaatgtgttattaacccttctcttcgaggacctgcaggtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatcgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatctcctgcaggatctggccggccggatctcgtacggatccgtcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgc

SEQ ID NO: 70 - DNA, 9616 bp, Artificial
  sequence of plasmid SH60
ggccgcgtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaagtgactcgaggtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaagtaactgcaggtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaagtagcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaatgtgttattaacccttctcttcgaggacctgcaggtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatcgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatctcctgcaggatctggccggccggatctcgtacggatccgtcgactctagaggccggccgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgc